(19) United States
(12) Patent Application Publication
(10) Pub. No.: US 2024/0245639 A1
(43) Pub. Date: Jul. 25, 2024

(54) Dermal Application of Retinoic Acid Receptor Agonists for Amelioration of Hypertrophic Scar

(71) Applicant: Northwestern University, Evanston, IL (US)

(72) Inventors: David Michael Dolivo, Evanston, IL (US); Adrian E. Rodrigues, Evanston, IL (US); Seok Jong Hong, Evanston, IL (US); Thomas A. Mustoe, Evanston, IL (US); Robert D. Galiano, Evanston, IL (US)

(21) Appl. No.: 18/422,823

(22) Filed: Jan. 25, 2024

Related U.S. Application Data
(60) Provisional application No. 63/481,569, filed on Jan. 25, 2023.

(51) Int. Cl. A61K 31/203; A61K 31/192; A61K 31/4535; A61P 17/02

(52) U.S. Cl. CPC A61K 31/203; A61K 31/192; A61K 31/4535; A61P 17/02

(57) ABSTRACT

Disclosed are compounds, compositions and methods for antagonizing fibroblast activation. Particularly disclosed are compounds, methods and compositions for treating and/or preventing scar formation with a retinoid.

### CROSS-REFERENCE TO RELATED APPLICATIONS

This patent application claims the benefit of priority of U.S. Provisional Patent Application No. 63/481,569, filed Jan. 25, 2023, which is incorporated herein by reference in its entirety.

### STATEMENT REGARDING FEDERALLY SPONSORED RESEARCH

This invention was made with government support under grant number AR081475 awarded by the National Institutes of Health. The government has certain rights in the invention.

### REFERENCE TO AN ELECTRONIC SEQUENCE LISTING

The contents of the electronic sequence listing (702581.02466.xml; Size: 23,832 bytes; and Date of Creation: Jan. 23, 2024) are herein incorporated by reference in its entirety.

### BACKGROUND OF THE INVENTION

Fibrosis denotes the process by which damaged tissue seeks to heal via deposition of a scar. A variety of injuries and insults can lead to the formation of fibrosis in numerous organs, resulting in nearly half of reported deaths in the industrialized world (Wynn, 2008). Therefore, therapeutic modalities that seek to prevent or treat fibrosis are critically needed. Different fibrotic diseases are characterized by distinct types of tissue injury, as well as by manifestations of tissue fibrosis that are specific to that condition. However, some cellular processes and pathological features appear to be common to all forms of fibrosis, and thus have often been the focus of anti-fibrotic drug development (Wynn, 2007). These features include the paradigm of the activated fibroblast, known as the myofibroblast, which is a major effector cell that leads to contraction of damaged tissue and deposition of mechanically aberrant, acellular collagenous tissue, known as scar tissue (Hinz, 2016).

Unfortunately, the complexity and redundancy inherent in the fibrotic response has complicated the successful development of anti-fibrotic therapeutics (Walraven and Hinz, 2018). Therefore, new targets and molecules with the potential to modulate the processes key to the development of fibrosis are of key interest for translational research.

### BRIEF SUMMARY OF THE INVENTION

Disclosed herein are compositions and methods for antagonizing fibroblast activation. One aspect of the present invention is a method of antagonizing fibroblast activation in a subject in need thereof comprising administering to the subject an effective amount of a retinoid. In some embodiments the fibroblast activation is in response to a wound or injury or the formation of a scar from a wound or injury. In some embodiments the retinoid comprises retinoic acid receptor agonist, such as CH 55 or all-trans retinoic acid. The retinoid may be administered dermally or by intradermal injection.

Another aspect of the present disclosure comprises a pharmaceutical composition for antagonizing fibroblast activation comprising an effective amount of a retinoid. In some embodiments the fibroblast activation is in response to a wound or injury or the formation of a scar from a wound or injury. In some embodiments the retinoid comprises retinoic acid receptor agonist, such as CH 55 or all-trans retinoic acid. The retinoid may be administered dermally or by intradermal injection.

Another aspect of the present disclosure comprises a method for treating scar formation comprising administering an effective amount of a retinoid. In some embodiments the retinoid is administered dermally or by intradermal injection. In some embodiments the retinoid is administered following closure of a wound. In some embodiments the retinoid comprises retinoic acid receptor agonist, such as CH 55 or all-trans retinoic acid.

Another aspect of the preset disclosure provides a method of antagonizing fibroblast activation comprising administering an effective amount of a checkpoint kinase inhibitor. In some embodiments the checkpoint kinase inhibitor comprises AZD-7762.

Another aspect of the present disclosure comprises a pharmaceutical composition for antagonizing fibroblast activation comprising an effective amount of a checkpoint kinase inhibitor.

In some embodiments the checkpoint kinase inhibitor comprises AZD-7762.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Pilot screen for anti-fibrotic effects in primary fibroblasts. (A) The gene expression signature of interest was extracted by taking the intersection of human LRRC15⁺ fibroblast-enriched marker genes (472 genes) and mouse Lrrc15⁺ fibroblast-enriched marker genes (523 genes), which yielded a common signature of 117 genes. This signature was used to query the CMap in order to predict small molecules to reverse the transcriptional paradigm. Five target compounds were then manually selected for screening in fibroblasts in vitro. (B,C) Primary human foreskin fibroblasts were grown in culture and exposed to vehicle control or indicated drugs at a single concentration for 24 hours. (B) Representative brightfield microscopy before (left) and after (right) treatment. Scale bar=100 μm. (C) Heatmap representing log₂fold change values of each replicate relative to mean expression of vehicle control replicates for indicated genes. GAPDH was used as an internal control. Veh=vehicle. AZD=AZD-7762. Ch55=Ch55. Pano=panobinostat. HHT=homoharringtonine. Eme=emetine. n=4 replicates per condition.

FIG. 2. Effects of Ch55 antagonistic to TGF-β1 stimulation in human fibroblasts. Primary human foreskin fibroblasts were grown in culture and exposed to vehicle control (Vehicle), vehicle+10 ng/mL rhTGF-β1 (TGF-β1), 1,000 nM Ch55 (Ch55), or 10 ng/mL rhTGF-β1+1,000 nM Ch55 (TGF-β1⁺ Ch55) in vitro. (A) Transcript quantification ofACTA2, CCN2, and SERPINE1 by qRT-PCR in cells harvested after 24 hours of treatment, expressed relative to vehicle-treated cells. GAPDH was used as an internal control. n=6 replicates per group. Statistical analysis was performed by one-way ANOVA followed by Tukey's post-hoc test, with selected statistical comparisons visualized in the figure. (B) Representative brightfield microscopy of fibroblasts from two donors immediately before harvest at 48 hours. Scale bar=100 μm. (C) Western blot analysis of samples in (B), analyzing expression of α-SMA and type I collagen. GAPDH was used as an internal control. (D,E) Representative immunostaining of (D) α-SMA or (E) Collagen I in cells treated and fixed at 48 hour harvest. F-actin was counterstained with Alexafluor-568-conjugated phalloidin, and nuclei were counterstained with DAPI. Scale bar=100 m.

FIG. 3. RNA-seq analysis of Ch55 effects in primary human foreskin fibroblasts. Primary human foreskin fibroblasts were grown in culture and exposed to vehicle control (Vehicle), vehicle+10 ng/mL rhTGF-β1 (TGF-β1), 1,000 nM Ch55 (Ch55), or 10 ng/mL rhTGF-β1+1,000 nM Ch55 (TGF-β1+Ch55) in vitro. RNA was harvested and expression profiling performed by RNA-seq. (A) PCA representation of variations in transcriptional profiles between and among treatment groups. (B,C) Heatmaps depicting normalized gene expression, represented as Z-scores, of Ch55-induced signatures (B) mimicking and (C) antagonizing TGF-β-induced effects. (D,E) Results of KEGG database query of signatures depicted in (B) and (C), respectively.

FIG. 4. Anti-fibrotic effects of Ch55 in a rabbit ear hypertrophic scar model in vivo. Rabbit ear excisional wounds were performed and treated with either Ch55 (high or low dose, 10 μg/wound or 2 μg/wound, respectively) or corresponding vehicle control via intradermal injections to closed wounds over the course of scar development, before terminating experiments on POD28. (A) Representative photographs of rabbit ear scars at time of harvest. (B) Visualization of representative hypertrophic scar cross-sections by H&E staining. Scale bar=1 mm. (C) Quantification of scar elevation index (SEI) for scar tissues at harvest. n=11-12 samples/condition. (D) Representative Western blot detecting expression of type I collagen in protein isolated from scar dermis at harvest for high dose and low dose Ch55 treatment groups and their respective controls. GAPDH was used as a loading control. (E) Densitometric quantification of type I collagen relative to GAPDH, as detected by Western blot. Density values for each sample are normalized to the mean of the respective vehicle controls for that dose. n=5-6 samples per group.

FIG. 5. Chemical structure of compounds selected from Connectivity Map analysis. Chemical line structures and CAS identification numbers of the five compounds identified from the CMap analysis and utilized in the initial screen.

FIG. 6. Concentration-dependence and generalizability of Ch55 treatment in human fibroblasts. Primary human foreskin fibroblasts were grown in culture and exposed to vehicle control or Ch55 at various concentrations for 24 hours. (A) Representative brightfield microscopy before (left) and after (right) treatment with Ch55 at indicated concentrations. Scale bar=100 μm. (B) Quantification of expression relative to vehicle control for indicated genes by qRT-PCR. n=4 replicates per condition. (C) Primary human foreskin fibroblasts from a second donor were cultured in the presence of vehicle or 1,000 nM Ch55 for 24 hours. Representative brightfield microscopy before (left) and after (right) treatment. Scale bar=100 μm. (D) Quantification of expression relative to vehicle control for indicated genes by qRT-PCR. n=6 replicates per condition.

FIG. 7. Ch55 effects on morphology and ACTA2 expression in rabbit dermal fibroblasts. Primary dermal fibroblasts isolated from two rabbits were grown in culture and exposed to vehicle control or 1,000 nM Ch55 for 24 hours. (A,C) Representative brightfield microscopy before (left) and after (right) treatment with Ch55 at indicated concentrations. Scale bar=100 μm. (B,D) Quantification of expression relative to vehicle control for ACTA2 by qRT-PCR. GAPDH was used as an internal control. n=4 replicates per condition.

FIG. 8. Effects of Ch55 on activation of human HTS and keloid fibroblasts. Primary human HTS and keloid fibroblasts were grown in culture and exposed to 1,000 nM Ch55 in the presence or absence of 10 ng/mL TGF-β1 for 48 hours prior to harvest. (A) Western blot analysis of samples for expression of type I collagen. GAPDH was used as an internal control. (B) Immunofluorescent staining for type I collagen. F-actin and nuclei were counterstained with rhodamine-conjugated phalloidin and DAPI, respectively. Scale bar=100 μm.

FIG. 9. Effects of ATRA on activation of human foreskin fibroblasts. Primary human foreskin fibroblasts were grown in culture and exposed to 10,000 nM ATRA in the presence or absence of 10 ng/mL TGF-β1 for 48 hours prior to harvest. (A) Western blot analysis of samples for expression of α-SMA and type I collagen. GAPDH was used as an internal control. (B,C) Immunofluorescent staining for (B) α-SMA and (C) type I collagen. F-actin and nuclei were counterstained with rhodamine-conjugated phalloidin and DAPI, respectively. Scale bar-100 μm.

FIG. 10. Transcriptional profiling of myofibroblast-related gene repression by Ch55. Relative expression of genes related to myofibroblast differentiation and related pro-fibrotic processes from RNA-seq data are depicted as Z-scores, calculated from extracted normalized count data.

FIG. 11. Transcriptional profiling of retinoic acid signaling-related gene dysregulation by Ch55. Relative expression of target or regulatory genes related to retinoic acid signaling from RNA-seq data are depicted as Z-scores, calculated from extracted normalized count data.

FIG. 12. Pathview visualization and DEGs for hsa04510: Focal adhesion. (A) Pathview visualization of the Focal adhesion KEGG term with Ch55-Vehicle gene expression profile overlaid. (B) Heatmap representation of TGF-β-antagonizing signature DEGs enriched in this pathway. Normalized gene expression values are depicted as Z-scores.

FIG. 13. Pathview visualization and DEGs for hsa04350: TGF-β signaling pathway. (A) Pathview visualization of the TGF-β signaling pathway KEGG term with Ch55-Vehicle gene expression profile overlaid. (B) Heatmap representation of TGF-β-antagonizing signature DEGs enriched in this pathway. Normalized gene expression values are depicted as Z-scores.

FIG. 14. Pathview visualization and DEGs for hsa04512: ECM-Receptor interaction. (A) Pathview visualization of the ECM-Receptor interaction KEGG term with Ch55-Vehicle gene expression profile overlaid. (B) Heatmap representation of TGF-β-antagonizing signature DEGs enriched in this pathway. Normalized gene expression values are depicted as Z-scores.

FIG. 15. Pathview visualization and DEGs for hsa04810: Regulation of actin cytoskeleton. (A) Pathview visualization of the Regulation of actin cytoskeleton KEGG term with Ch55-Vehicle gene expression profile overlaid. (B) Heatmap representation of TGF-β-antagonizing signature DEGs enriched in this pathway. Normalized gene expression values are depicted as Z-scores.

FIG. 16. Effects of Ch55 treatment on erythema in rabbit ear hypertrophic scars in vivo. Spectroscopic readings were performed to assess erythema at POD28 harvest of developed rabbit ear hypertrophic scars using a DermaLab Combo. All values are corrected by subtracting background readings from uninjured tissue in the center of the same ear, distant from wound locations. n=12 samples/condition.

FIG. 17. Cartoon depiction of scar elevation index (SEI). Schematic diagram for SEI determination from histological cross-sections.

FIG. 18. Histological collagen stains of rabbit HTS dermis. Representative histological stains of rabbit HTS tissues at POD28 as described in FIG. 4 stained with (A) Masson's trichrome or (B) picrosirius red/fast green. Scale bar=100 μm.

FIG. 19. RAR family transcript expression in human foreskin fibroblasts. Expression of RARA/B/G transcripts extracted from RNA-seq data presented in FIG. 3. (A) Pie chart representing relative proportions of each RAR transcript per group of samples, as assessed by FPKM values. (B) FPKM values of each RAR transcript for each group of samples represented in bar graphs, grouped by (left) transcript and (right) treatment condition.

### DETAILED DESCRIPTION OF THE INVENTION

Fibrosis denotes the process by which damaged tissue seeks to heal via deposition of a scar. A variety of injuries and insults can lead to the formation of fibrosis in numerous organs, resulting in nearly half of reported deaths in the industrialized world. Therefore, therapeutic modalities that seek to prevent or treat fibrosis are critically needed. Described herein are methods and compositions for antagonizing fibrosis activation.

In a first aspect of the invention, a method of antagonizing fibroblast activation in a subject in need, the method comprising administering to a subject an effective amount of a retinoid is provided.

A fibroblast is a type of cell that contributes to the formation of connective tissue. Fibrocytes are circulating fibroblast-like cells in the vascular system that are derived from bone marrow stem cells. “Fibrocyte” is a term sometimes ascribed to a relatively inactive fibroblast-like cell, whereas the term “fibroblast” designates a fully active cell. As used herein, “fibroblast” “fibrocyte” are used interchangeably. Active fibroblast synthesize and secrete extracellular matrix and collagen proteins that help maintain the structural framework of tissues. Fibroblasts respond to wound healing by chemotaxing and proliferating to the sites of tissue injury to rebuild the extracellular matrix as a scaffold for tissue regeneration. Fibroblast to myofibroblast transitioning enables the contraction of the matrix to seal an open wound in the event of the loss of tissue. Stimuli that initiate fibroblast activation mostly derive from macrophages. Activation of fibroblasts include proliferation, fibrinogenesis, and release of cytokine and proteolytic enzymes. Antagonizing fibroblast activation blocks, inhibits, reduces or prevents the function of fibroblasts. Markers of fibroblast activation include genes related to fibroblast function, including but not limited to Actin alpha 2, smooth muscle (ACTA2), Calponin 2 (CCN2), Matrix metallopeptidase 1 (MMP1), serine proteinase inhibitor family E member 1 (SERPINE1), Fibronectin 1 (FN1), Transforming Growth factor beta 1 (TGFB1), Collagen type I alpha 2 chain (COL1A2), Lysyl oxidase-like 1 (LOXL1), Calponin 3 (CNN3), Thy-1 cell surface antigen (THY1) and those genes listed in Table 2. In some embodiments decreased activation of fibroblasts can be evaluated by the gene or protein expression of ACTA2, CNN1, CCN2, SERPINE1, TAGLN, EDN1, and IL6. Indicators of fibroblast activation may further comprise erythema in developed scars, scar elevation index, hypertrophy or the amount of type I collagen at the site.

As used herein, antagonizing with respect to a method of antagonizing fibroblast activation means to inhibit or stop the action of another. Antagonizing may be used interchangeable with antagonist. A method of antagonizing may include interfering with, preventing, blocking or reducing the physiological action of another. An antagonist may be reversible or irreversible. A method of antagonizing fibroblast activation is described herein, wherein fibroblast activation is decreased.

The proper synthesis and degradation of extracellular matrix ensure normal tissue architecture is preserved after a wound or injury. However, if the wound or injury is severe, repetitive or if the healing response itself becomes dysregulated a pathological accumulation of extracellular matrix can occur. This pathological response is fibrosis. Fibrosis, also known as fibrotic scarring, is a pathological wound healing in which connective tissue replaces normal parenchymal tissue to the extent that it goes unchecked, leading to considerable tissue remodeling and the formation of permanent scar tissue. Scar tissue is a collection of cells and collagen that covers the site of the injury. A scar or scar tissue can be keloid, a hypertrophic scar or a contracture scar. Fibrosis can occur in many tissues within the body, including but not limited to lungs, liver, kidney, brain, heart. Examples provided herein demonstrate fibrosis within the skin. In some embodiments fibroblast activation is in response to a wound or injury. In additional embodiments the wound or injury results in the formation of scar tissue or fibrosis. Types of wounds include, but are not limited to penetrating, open wounds (puncture, laceration, abrasion, avulsion, surgical, incision, thermal, chemical, electrical, bites, or from high velocity projectiles), and blunt force trauma.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. In some embodiments, the method comprises dermal administration. Dermal administration delivers adequate concentrations of a composition to the right localization in the skin, where it should remain for a sufficient time period. Other topical formulations include aerosols, conditioners, solutions (gels, ointments, creams, and suspensions), bandages and other wound dressings. Alternatively, one may incorporate or encapsulate the composition in a suitable polymer matrix or membrane, thus providing a sustained-release delivery device suitable for implantation near the site to be treated locally. In some embodiments, compositions described herein may be administered following closure of a wound. Wound closure may take place via primary intention for example with sutures, staples or tape, or via secondary intention with secondary healing.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be reducing the activation of fibroblasts.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A “subject in need thereof” as utilized herein may refer to a subject in need of treatment for a disease or disorder associated with activated fibroblasts, fibrosis or scarring. The term “subject” may be used interchangeably with the terms “individual” and “patient” and includes human and non-human mammalian subjects. A subject may include those with hypertrophic scars including burn scars, keloids, scleroderma, and fibrosis resulting from graft-versus-host disease or from radiation. A subject may also include those with lung fibrosis, liver fibrosis, kidney fibrosis, glial scars, myocardial fibrosis or other forms of organ or tissue fibrosis or adhesions. Subject may also include individuals who have undergone surgery. Methods and or compositions provided herein may be administered following surgery following the closure of a wound.

Methods and compositions described herein may be appropriate for subjects with prior scarring history including those with a history of keloid or hypertrophic scarring or those with a genetic predisposition or genetic history of scarring. Subjects with wounds characteristics that are likely to lead to scarring, including the depth or severity of the wound and location of the wound as well as the health of the subject, may also be treated with methods and compositions described herein. Methods and compositions described herein may also be used for cosmetic reasons, including following surgery or following a severe wound.

In some embodiments, a retinoid is administered. Retinoids are a class of chemical compounds that are vitamers of vitamin A or are chemically related to it. There are four generations of retinoids, first generation retinoids include retinol, retinal, tretinoin (retinoic acid), isotretinoin, and alitretinoin, second generation retinoids include etretinate and its metabolite acitretin, third generation retinoids include adapalene, bexarotene, and tazarotene and fourth generation retinoids includes Trifarotene.

In some embodiments, the retinoid includes CH 55. Ch 55 (CAS No: 110368-33-7) is a highly potent synthetic retinoid that has high affinity for RAR-α and RAR-β receptors and low affinity for cellular retinoic acid binding protein (CRABP).

In some embodiments, the retinoid is a retinoic acid receptor (RAR) agonist. RAR is a nuclear receptor which can also act as a ligand-activated transcription factor. RAR is active by both all-trans retinoic acid and 9-cis retinoic acid among other agonists. There are three retinoic acid receptors, RAR-alpha, RAR-beta, and RAR-gamma, encoded by the RARA, RARB, RARG genes, respectively. In some embodiments, the RAR agonist includes all trans retinoic acid. Other RAR agonists include, but are not limited to, AC 261066 (CAS No. 870773-76-5), Adapalene (CAS No. 106685-40-9), AM 580 (CAS No. 102121-60-8), AM 80 (CAS No. 94497-51-5), BMS 753 (CAS No. 215307-86-1), BMS 961 (CAS No. 185629-22-5), CD 1530 (CAS No. 107430-66-0), CD 2314 (CAS No. 170355-37-0), CD 437 (CAS No. 125316-60-1), DC 271 (CAS No. 198696-03-6) and TTNPB (CAS No. 71441-28-6).

Another aspect of the present disclosure comprises a pharmaceutical composition for antagonizing fibroblast activation, the composition comprising an effective amount of a retinoid.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration. Alternatively, dermal or transdermal delivery systems manufactured as an adhesive disc or patch which slowly releases the compound(s) for percutaneous absorption may be used. To this end, permeation enhancers may be used to facilitate transdermal penetration of the compound(s).

In some embodiments the pharmaceutical composition may be delivered dermally, and may comprise CH 55.

In some embodiments the pharmaceutical composition may be delivered by intradermal injection, and may comprise CH 55.

Another aspect of the present disclosure comprises a method of treating scar formation, the method comprising administering a retinoid. In some injuries the fibrosis of the wound occurs before the restoration of normal tissue structure can be completed, thus irreversible scar tissue is formed. A scar can be a fine-line scar, a keloid scar, a hypertrophic scar, a pitted or sunken scar, a atrophic scar, a contracture scar or a stretch mark.

In some embodiments a method for antagonizing fibroblast activation comprises administering an effective amount of a checkpoint kinase inhibitor. In some embodiments AZD-7762 is administered. AZD-7762 (CAS Number: 1246094-78-9) is a checkpoint kinase 1 and 2 (CHK1/2) inhibitor. Some embodiments comprise a pharmaceutical composition for antagonizing fibroblast activation, the composition comprising an effective amount of a checkpoint kinase inhibitor or AZD-7762.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1—Prediction and Demonstration of Retinoic Acid Receptor Agonist Ch55 as an Anti-Fibrotic Agent in the Dermis Myofibroblast Gene Expression Profile Predicts Potential Anti-Fibrotic Compounds

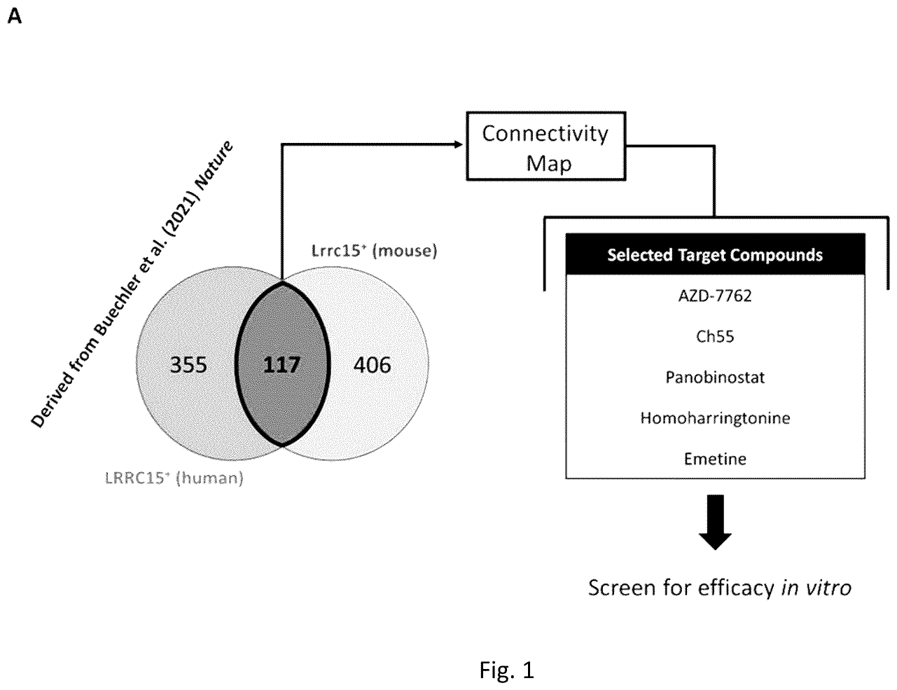

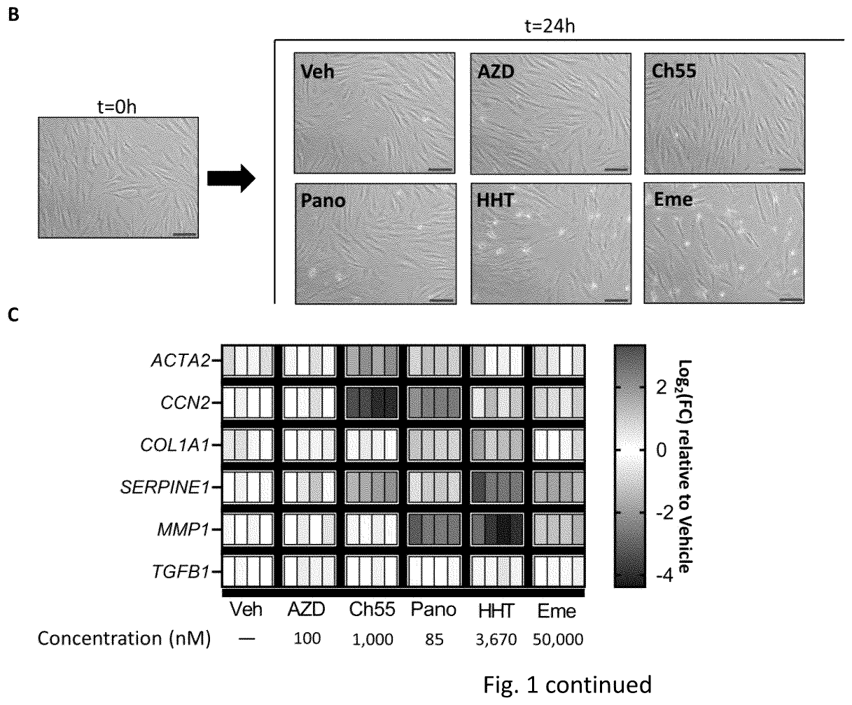

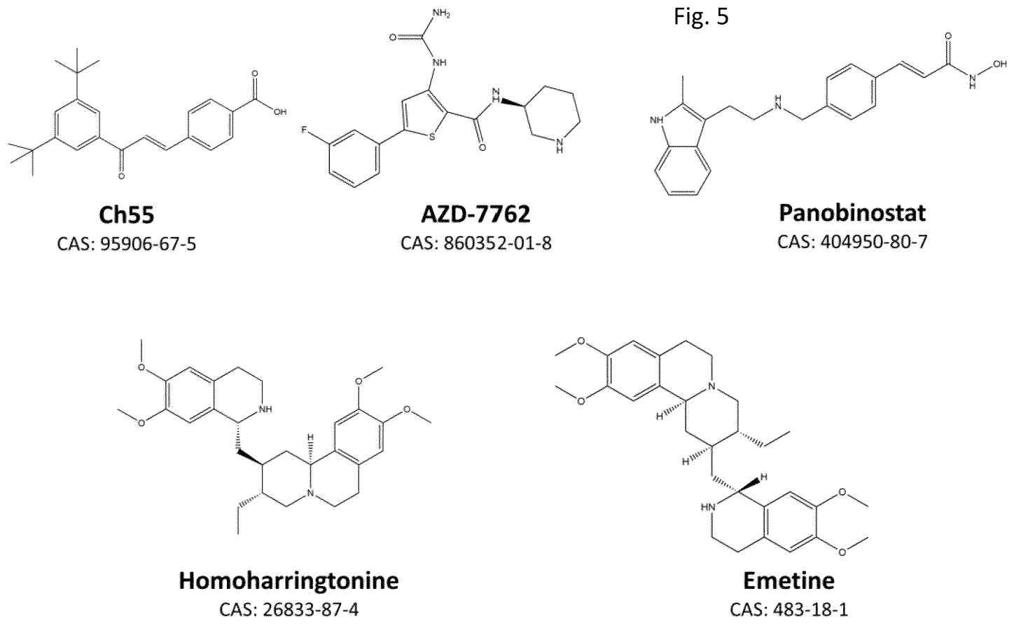

The activity of myofibroblasts is a causative factor underlying all fibrotic pathological states. Activation of myofibroblasts is characterized by dysregulation of common and unique sets of genes in fibroblasts or in other cells that can differentiate into myofibroblasts, depending on the tissue in question (Hinz et al., 2007). We hypothesized that a set of genes commonly upregulated across myofibroblasts irrespective of origin might be a useful signature to predict compounds that antagonize myofibroblast activation and, therefore, suppress fibrosis. Recently, Buechler et al. (Buechler et al., 2021) used single-cell RNA-seq to characterize a conserved myofibroblast population found across several pathological states in varied tissues and organs in humans (LRRC15⁺) and in mice (Lrrc15⁺). In order to maximize generalizability of predictions made using this signature, we extracted the intersection of the overexpressed gene sets in both human and mouse, yielding a combined signature of 117 genes (FIG. 1A, genes and their encoded proteins listed in Table 1). Subjecting this gene signature to CMap analysis (accessed at clue.io, (Subramanian et al., 2017)) yielded a list of compounds predicted to reverse this gene expression signature. Several high-ranking compounds in this list included known TGF-βR inhibitors (SB-431542, LY-2157299, LY-364947, and D-4476), the DPP4 inhibitor sitagliptin, and the clinically approved anti-fibrotic tyrosine kinase inhibitor nintedanib, among many other compounds with myriad known targets and/or mechanisms of action. The well-characterized roles in fibrosis of the targets of several of these drugs, such as TGF-βRs, lent greater confidence to the ability of this screen to reveal other compounds with anti-fibrotic activity as well. We chose five high-ranking compounds from the list without connections to tissue fibrosis and decided to further investigate their potential as antifibrotic agents (FIG. 1A). Chemical structures of these compounds and their corresponding CAS identifiers are presented in FIG. 5.

TABLE 1
List of genes comprising the LRRC15+/Lrrc15+
gene expression signature

| Gene Symbol | Protein Encoded |
|---|---|
| ACTA2 | Actin alpha 2, smooth muscle |
| ACTB | Actin beta |
| ACTN1 | Actinin alpha 1 |
| ADAM12 | ADAM metallopeptidase domain 12 |
| AEBP1 | AE binding protein 1 |
| AK1 | Adenylate kinase 1 |
| ANGPTL2 | Angiopoietin like 2 |
| ANTXR1 | ANTXR cell adhesion molecule 1 |
| ARL4C | ADP ribosylation factor like GTPase 4C |
| ASPN | Asporin |
| BASP1 | Brain abundant membrane attached signal protein 1 |
| BGN | Biglycan |
| BMP1 | Bone morphogenetic protein 1 |
| C1QTNF3 | C1q and TNF related 3 |
| C1QTNF6 | C1q and TNF related 6 |
| CALD1 | Caldesmon 1 |
| CALU | Calumenin |
| CKAP4 | Cytoskeleton associated protein 4 |
| CNN2 | Calponin 2 |
| CNN3 | Calponin 3 |
| COL12A1 | Collagen type XII alpha 1 chain |
| COL1A1 | Collagen type I alpha 1 chain |
| COL1A2 | Collagen type I alpha 2 chain |
| COL5A2 | Collagen type V alpha 2 chain |
| COL8A1 | Collagen type VIII alpha 1 chain |
| CREB3L1 | cAMP responsive element binding protein 3 like 1 |
| CRTAP | Cartilage associated protein |
| CSRP2 | Cysteine and glycine rich protein 2 |
| CTHRC1 | Collagen triple helix repeat containing 1 |
| CTNNB1 | Catenin beta 1 |
| CTSK | Cathepsin K |
| CXCL2 | C-X-C motif chemokine ligand 2 |
| DAP | Death associated protein |
| EEF1G | Eukaryotic translation elongation factor 1 gamma |
| EFEMP2 | EGF containing fibulin extracellular matrix protein 2 |
| FBLN2 | Fibulin 2 |
| FIBIN | Fin bud initiation factor homolog |
| FKBP10 | FKBP prolyl isomerase 10 |
| FKBP9 | FKBP prolyl isomerase 9 |
| FN1 | Fibronectin 1 |
| FNDC1 | Fibronectin type III domain containing 1 |
| FOSB | FosB proto-oncogene, AP-1 transcription factor subunit |
| FOXP1 | Forkhead box P1 |
| FSCN1 | Fascin actin-bundling protein 1 |
| FZD1 | Frizzled class receptor 1 |
| GABARAP | GABA type A receptor-associated protein |
| GAPDH | Glyceraldehyde-3-phosphate dehydrogenase |
| GPC1 | Glypican 1 |
| GPX7 | Glutathione peroxidase 7 |
| HCFC1R1 | Hose cell factor C1 regulator 1 |
| INHBA | Inhibin subunit beta A |
| ITGB5 | Integrin subunit beta 5 |
| ITGBL1 | Integrin subunit beta like 1 |
| KDELR2 | KDEL endoplasmic reticulum protein retention receptor 2 |
| KDELR3 | KDEL endoplasmic reticulum protein retention receptor 3 |
| LOXL1 | Lysyl oxidase like 1 |
| LOXL2 | Lysyl oxidase like 2 |
| LRRC15 | Leucine rich repeat containing 15 |
| LUM | Lumican |
| MAGED1 | MAGE family member D1 |
| MARCKSL1 | MARCKS like 1 |
| MDK | Midkine |
| MFAP2 | Microfibril associated protein 2 |
| MIF | Macrophage migration inhibitory factor |
| MMP14 | Matrix metallopeptidase 14 |
| MMP19 | Matrix metallopeptidase 19 |
| MMP2 | Matrix metallopeptidase 2 |
| MORF4L2 | Mortality factor 4 like 2 |
| MYH9 | Myosin heavy chain 9 |
| MYL9 | Myosin light chain 9 |
| NR4A2 | Nuclear receptor subfamily 4 group A member 2 |
| NREP | Neuronal regeneration related protein |
| NXN | Nucleoredoxin |
| OLFML2B | Olfactomedin like 2B |
| OLFML3 | Olfactomedin like 3 |
| P3H1 | Prolyl 3-hydroxylase 1 |
| P3H4 | Prolyl 3-hydroxylase family member 4 |
| P4HA3 | Prolyl 4-hydroxylase subunit alpha 3 |
| P4HB | Prolyl 4-hydroxylase subunit beta |
| PABPC1 | Poly(A) binding protein cytoplasmic 1 |
| PALLD | Palladin, cytoskeletal associated protein |
| PCSK5 | Proprotein convertase subtilisin/kexin type 5 |
| PDGFRL | Platelet derived growth factor receptor like |
| PDLIM7 | PDZ and LIM domain 7 |
| PMEPA1 | Prostate transmembrane protein, androgen induced 1 |
| POSTN | Periostin |
| PPIC | Peptidylprolyl isomerase C |
| PTK7 | Protein tyrosine kinase 7 |
| RAB31 | RAB31, member RAS oncogene family |
| RCN3 | Reticulocalbin 3 |
| RGS3 | Regulator of G protein signaling 3 |
| SCARF2 | Scavenger receptor class F member 2 |
| SDC1 | Syndecan 1 |
| SFRP2 | Secreted frizzled related protein 2 |
| SKIL | SKI like proto-oncogene |
| SLC39A14 | Solute carrier family 39 member 14 |
| SMCO4 | Single-pass membrane protein with coiled-coil domains 4 |
| SMIM3 | Small integral membrane protein 3 |
| SNHG18 | Small nucleolar RNA host gene 18 |
| SPARC | Secreted protein acidic and cysteine rich |
| SPATS2L | Spermatogenesis associated serine rich 2 like |
| SPON1 | Spondin 1 |
| SRPX2 | Sushi repeat containing protein X-linked |
| SSR3 | Signal sequence receptor subunit 3 |
| SULF2 | Sulfatase 2 |
| TAGLN | Transgelin |
| THBS2 | Thrombospondin 2 |
| THY1 | Thy-1 cell surface antigen |
| TMEM119 | Transmembrane protein 119 |
| TMEM263 | Transmembrane protein 263 |
| TNFAIP3 | TNF alpha induced protein 3 |
| TPBG | Trophoblast glycoprotein |
| TPM1 | Tropomyosin 1 |
| TUBB2B | Tubulin beta 2B class iib |
| VCAN | Versican |
| WISP1 | WNT1 inducible signaling pathway protein 1 |
| YWHAZ | Tyrosine 3-monooxygenase/tryptophan 5-monooxygenase |
| | activation protein zeta |

### In Vitro Screen of Predicted Compounds Indicates Ch55 as a Potential Molecule of Interest

In order to initially test the identified drugs, we applied each drug or DMSO vehicle control at a single concentration to primary human foreskin fibroblasts in vitro for 24 hours, before harvesting and measuring relative expression of selected myofibroblast marker genes by qRT-PCR. The checkpoint kinase inhibitor AZD-7762 (Mitchell et al., 2010), the retinoic acid receptor agonist Ch55 (Li Xiang et al., 2017, Ye et al., 2016), the histone deacetylase inhibitor panobinostat (Korfei et al., 2018), the protein synthesis inhibitor homoharringtonine (Li Xiaolei et al., 2017, Sun et al., 2021), and the protein synthesis inhibitor emetine (Yang et al., 2018) were applied based on concentrations previously demonstrated to have effects on cultured cells in vitro. Culture of human foreskin fibroblasts in the presence of emetine or homoharringtonine resulted in notable detachment of cells from the tissue culture plate by the time of harvest, while none of the other drugs yielded obvious cellular detachment at the tested concentrations (FIG. 1B). Quantification of relative expression of some common myofibroblast and fibrosis-associated genes (Pakshir et al., 2020, Samarakoon et al., 2013) (FIG. 1C) revealed that emetine and homoharringtonine, in addition to their detachment effects, also induced myofibroblast activation, as assessed by increased expression of CCN2 (the gene encoding connective tissue growth factor), increased expression of SERPINE1 (the gene encoding plasminogen activator inhibitor 1), and decreased expression of MMP1 (the gene encoding matrix metalloproteinase 1), a signature also shared by panobinostat. While AZD-7762 did not show a similar effect of fibroblast activation, neither did it appear to downregulate myofibroblast-associated genes. In contrast, application of Ch55 resulted in clear and consistent downregulation of ACTA2 (the gene encoding smooth muscle α-actin), CCN2, and SERPINE1 across replicates, without downregulating MMP1, compared to expression in vehicle-treated fibroblasts. None of the drugs tested appeared to affect expression of TGFB1 (the gene encoding transforming growth factor-beta 1) at the examined concentrations. Taken together, these data suggested that Ch55 was a putative anti-fibrotic molecule worthy of further investigation.

### Ch55-Mediated Antagonism of Fibroblast Activation Indicates a Concentration-Dependent Response and is not Donor-Specific

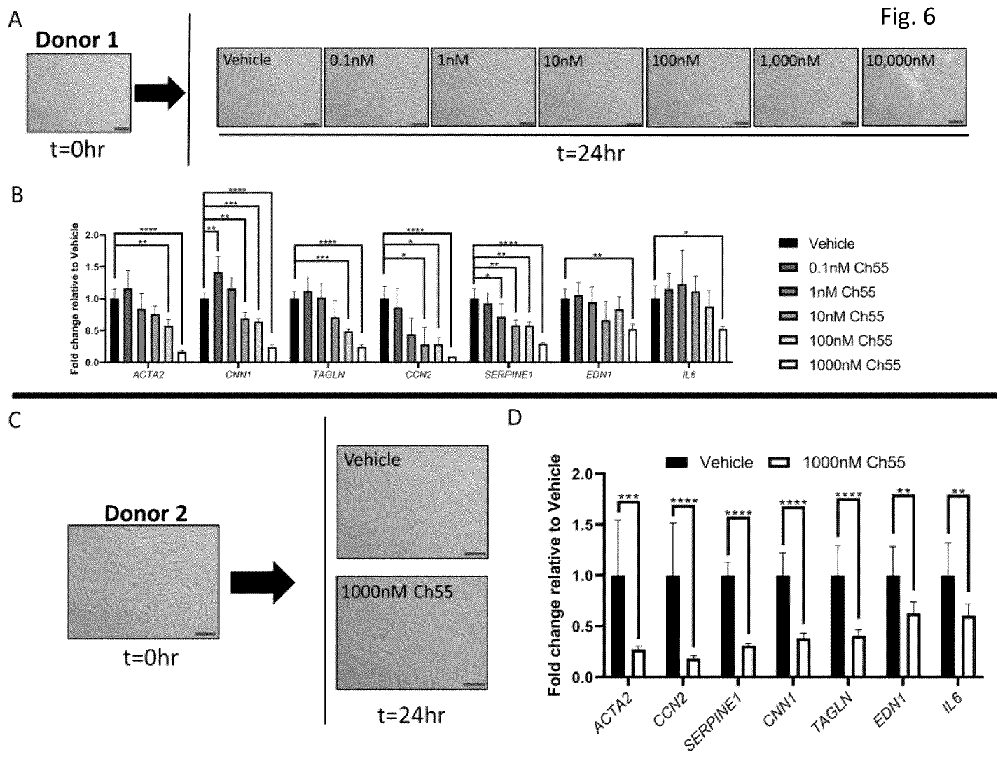

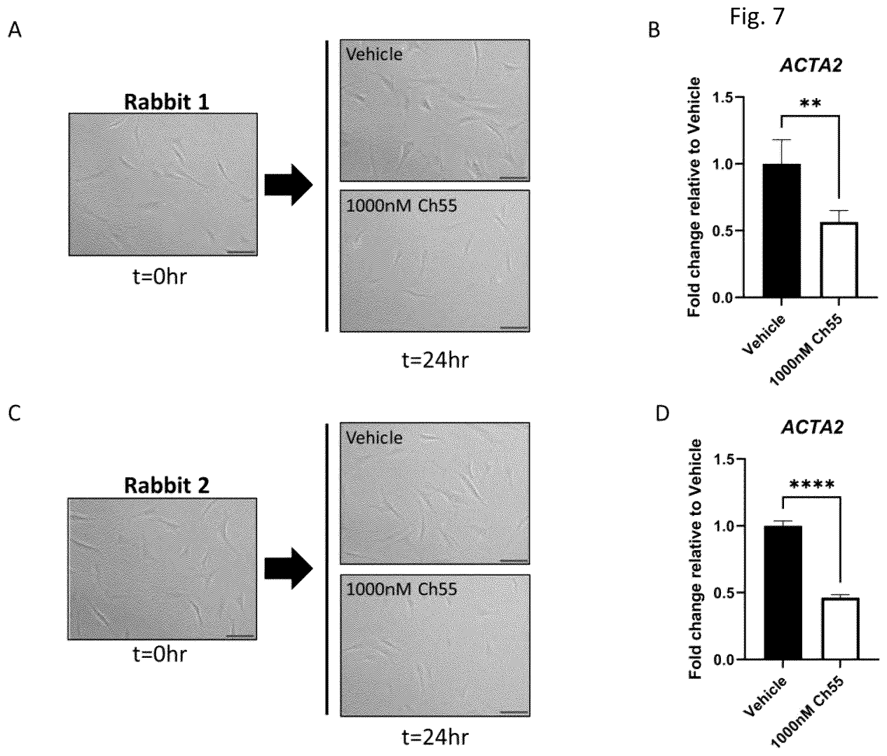

We next sought to understand the concentration range over which Ch55 was active in fibroblasts. We treated primary human foreskin fibroblasts for 24 hours with DMSO vehicle control or log₁₀ diluted concentrations of Ch55 from 10,000 nM to 0.1 nM. While no obvious cellular detachment was observed in fibroblasts treated with 0-1,000 nM Ch55, consistent with our initial experiment presented in FIG. 1B, treatment with 10,000 nM Ch55 resulted in near complete detachment of fibroblasts from the tissue culture plate (FIG. 6A). Analysis of expression of several myofibroblast markers and pro-fibrotic genes by qRT-PCR demonstrated concentration dependence of the antagonistic effects of Ch55. This effect was maximal at 1,000 nM among tested concentrations, but significant decreases in expression of ACTA2 and TAGLN (the gene encoding transgelin) were also demonstrated at 100 nM, CCN2 and CNN1 (the gene encoding calponin 1) as low as 10 nM, and SERPINE1 as low as 1 nM, while the decrease in expression of IL6 was only statistically significant at 1,000 nM (FIG. 6B). RNA was not harvested from cells treated with 10,000 nM Ch55 due to near-complete cell detachment after 24 hours of treatment. Treatment of primary human foreskin fibroblasts isolated from another tissue donor yielded comparable effects (FIG. 6C,D), as did treatment of primary rabbit dermal fibroblasts (FIG. 7A-D), suggesting that these effects were not due to a donor-specific idiosyncrasy.

### Ch55 Maintains Potential to Antagonize Fibroblast Activation in the Presence of TGF-β1

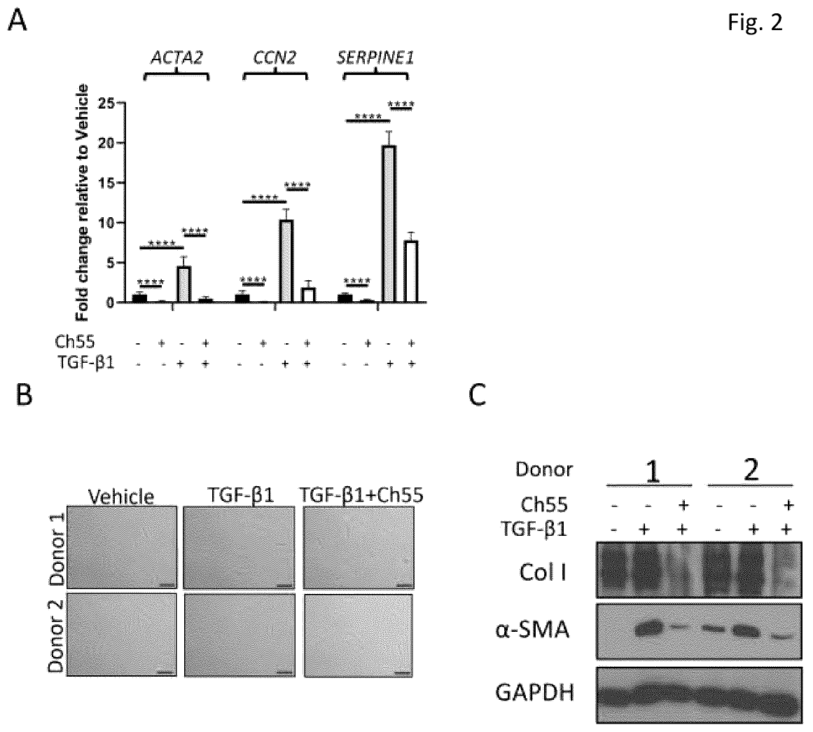

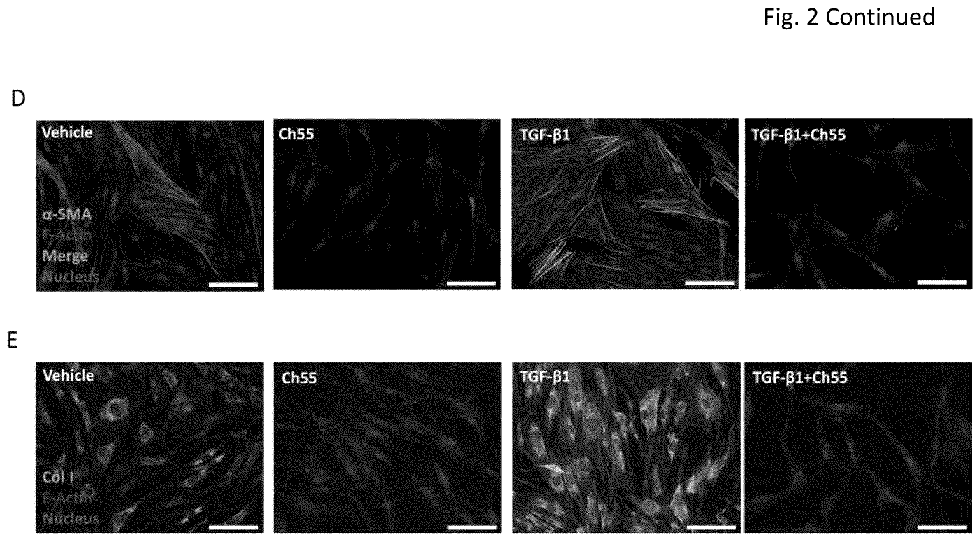

Since myofibroblast activation occurs in the context of active TGF-θ signaling, we next determined whether Ch55 maintained its effectiveness to antagonize fibroblast activation and collagen deposition in fibroblasts treated with TGF-β1. Primary human foreskin fibroblasts were cultured in the presence of vehicle control, TGF-β1, Ch55, or TGF-β1 and Ch55. At 24 hour harvest, analysis by qRT-PCR demonstrated that treatment with Ch55 significantly downregulated both basal and TGF-β1-induced expression of ACTA2, CCN2, and SERPINE1 (FIG. 2A). Culture for 48 hours demonstrated that Ch55 appeared to antagonize fibroblast activation, with decreased spread area per cell, as assessed by brightfield microscopy (FIG. 2B). Western blot analysis of lysates prepared from these samples at harvest confirmed that Ch55 dramatically antagonized fibroblast activation and collagen deposition as assessed by expression of α-SMA and type I collagen (FIG. 2C). Immunofluorescent staining and analysis revealed that Ch55 treatment resulted in loss of α-SMA protein from filamentous actin stress fibers (FIG. 2D), as well as greatly diminished deposition of collagen I (FIG. 2E), in both the presence and absence of exogenous TGF-β1.

Ch55 Reduces Collagen Deposition in Fibroblasts Sourced from Hypertrophic Scar and Keloid

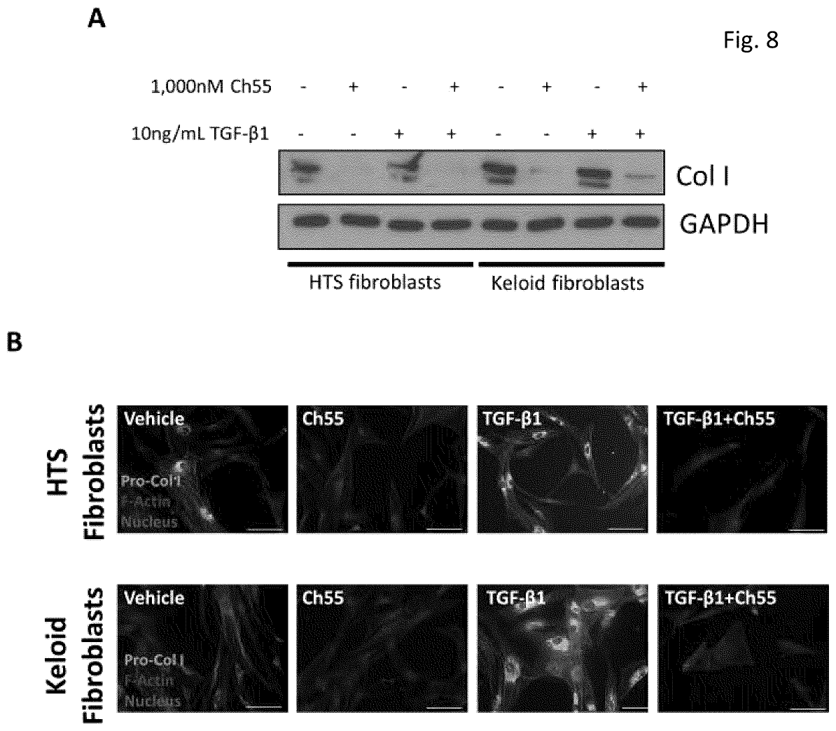

Since overproduction of collagen is characteristic of fibroblasts in tissue fibrosis, including hypertrophic scar and keloid, we wished to determine whether Ch55 also affects deposition of collagen I by these fibroblasts. Analysis of collagen I expression by Western blot (FIG. 8A) and immunofluorescence (FIG. 8B) demonstrated dramatic reduction of collagen I deposition by Ch55 in HTS and keloid fibroblasts, both in the presence and absence of exogenous TGF-β1. This suggests that Ch55 is also effective at reducing collagen deposition in pathological scar fibroblasts.

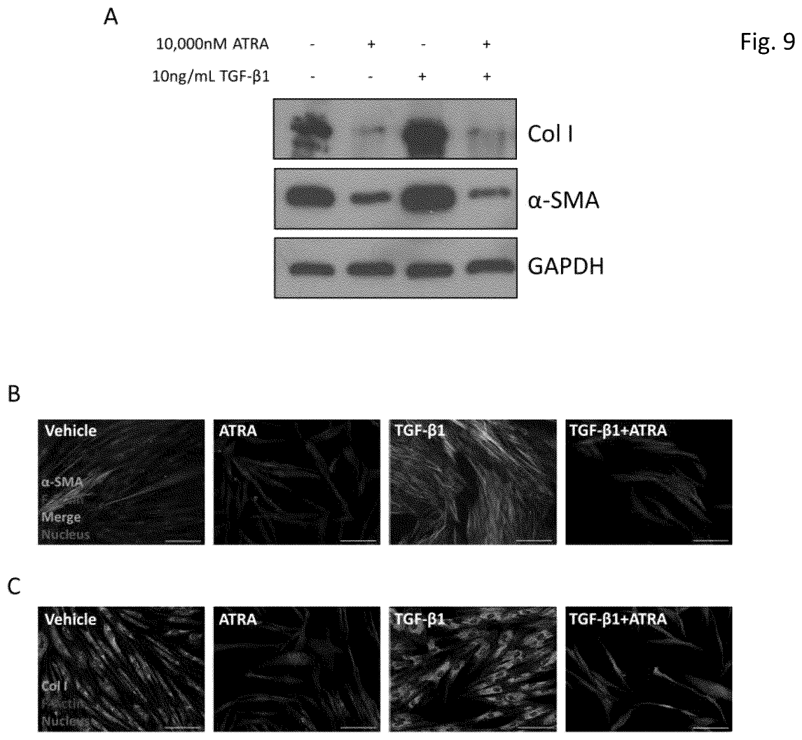

RAR Agonist all-Trans Retinoic Acid Antagonizes Myofibroblast Activation We then wished to see whether another RAR agonist could antagonize myofibroblast activation. Treatment of primary human foreskin fibroblasts with 10,000 nM all-trans retinoic acid (ATRA) substantially decreased α-SMA and collagen I protein, as well as F-actin stress fiber formation, as determined by Western blot (FIG. 9A) and immunofluorescence (FIG. 9B,C). Treatment with ATRA also dramatically decreased expression of collagen I in primary human fibroblasts isolated from HTS and keloid (data not shown). This suggests that the effects of Ch55 antagonistic to fibroblast activation and collagen deposition are not unique to Ch55 among RAR agonists.

### Ch55-Dysregulated Transcriptional Paradigms Antagonistic to TGF-α1 Stimulation are Enriched for Pathways Relevant to Tissue Fibrosis

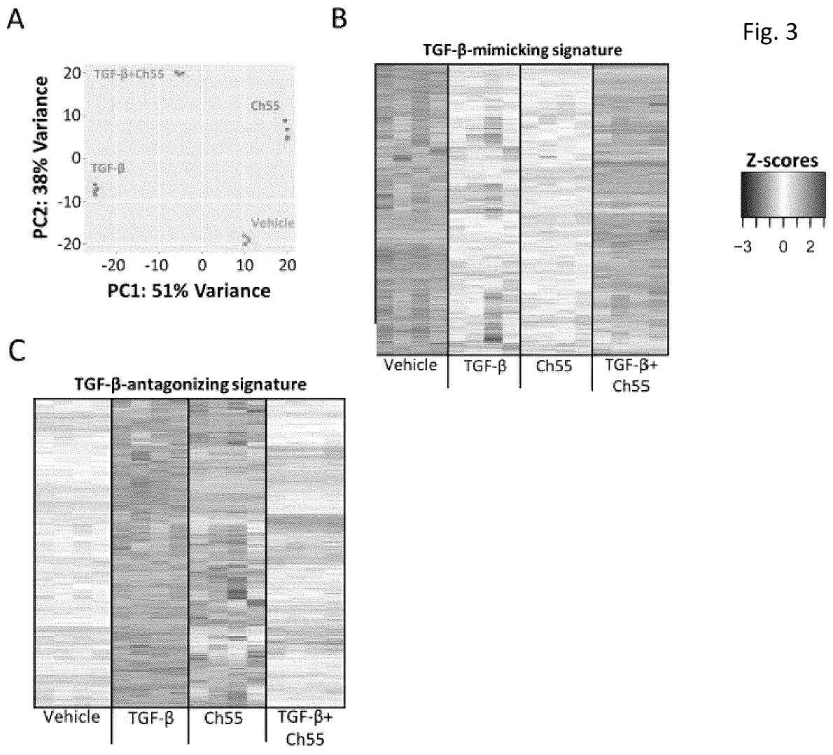

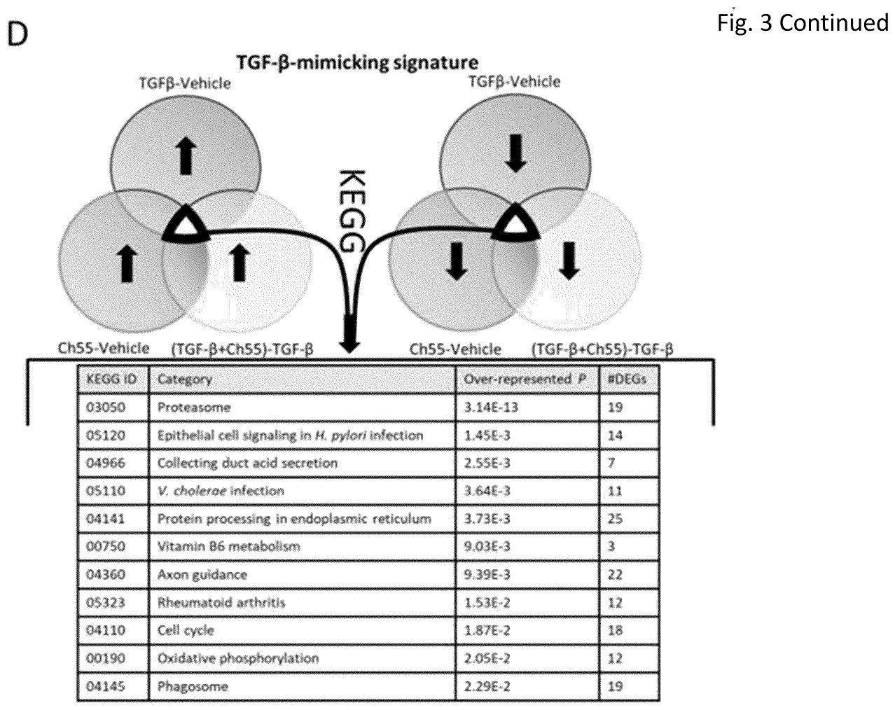

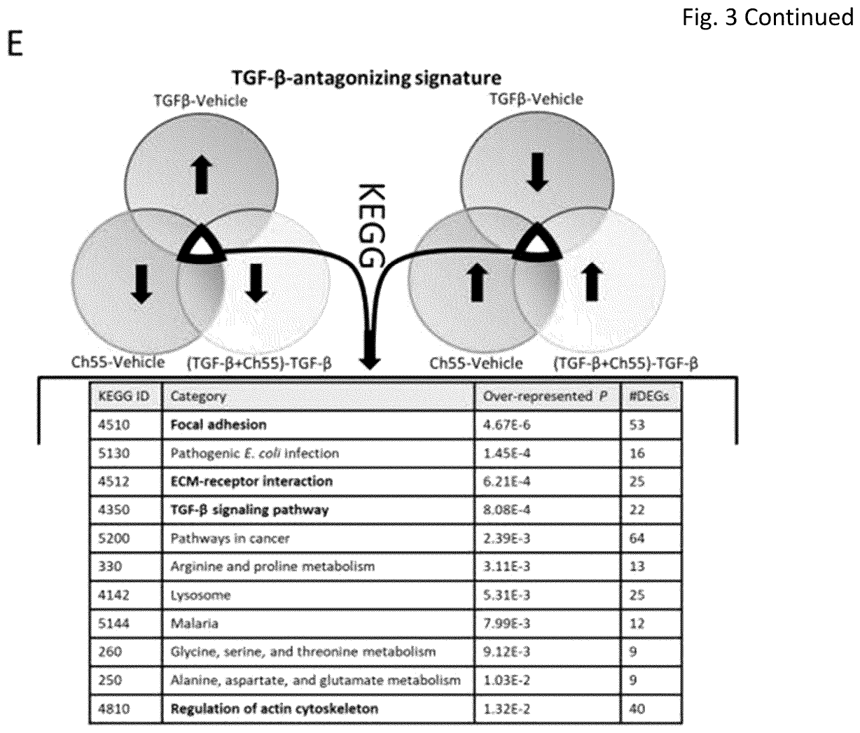

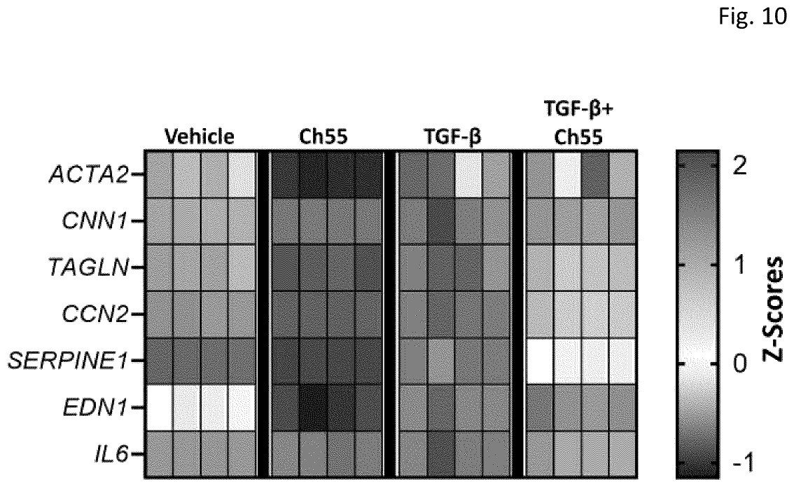

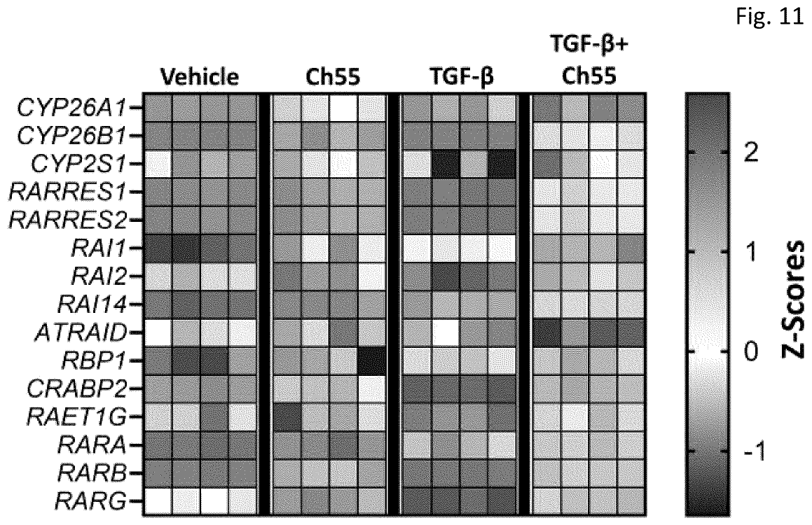

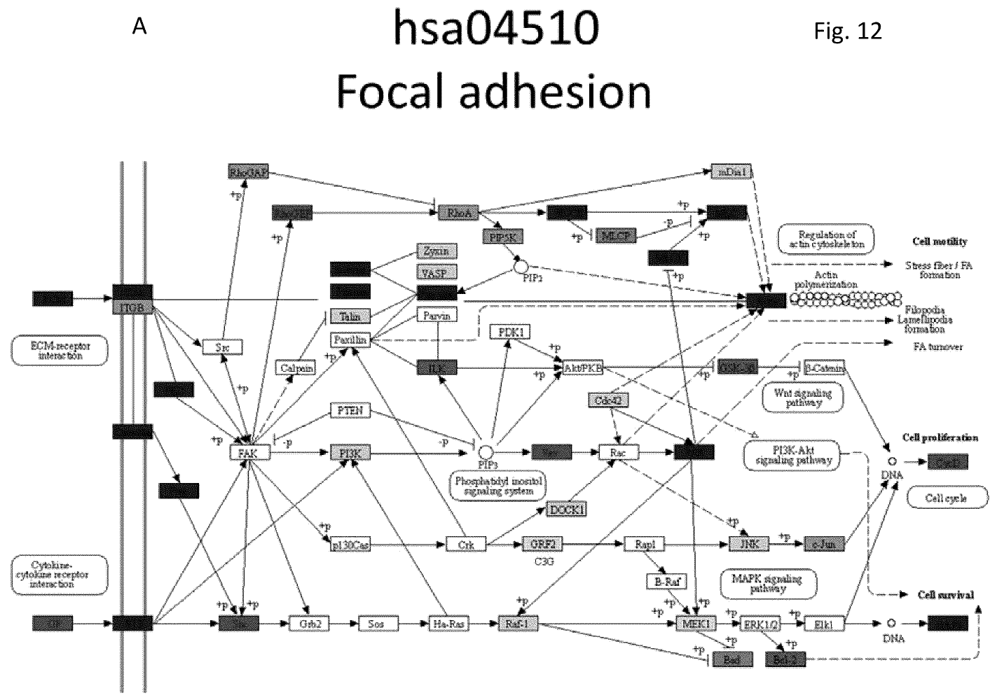

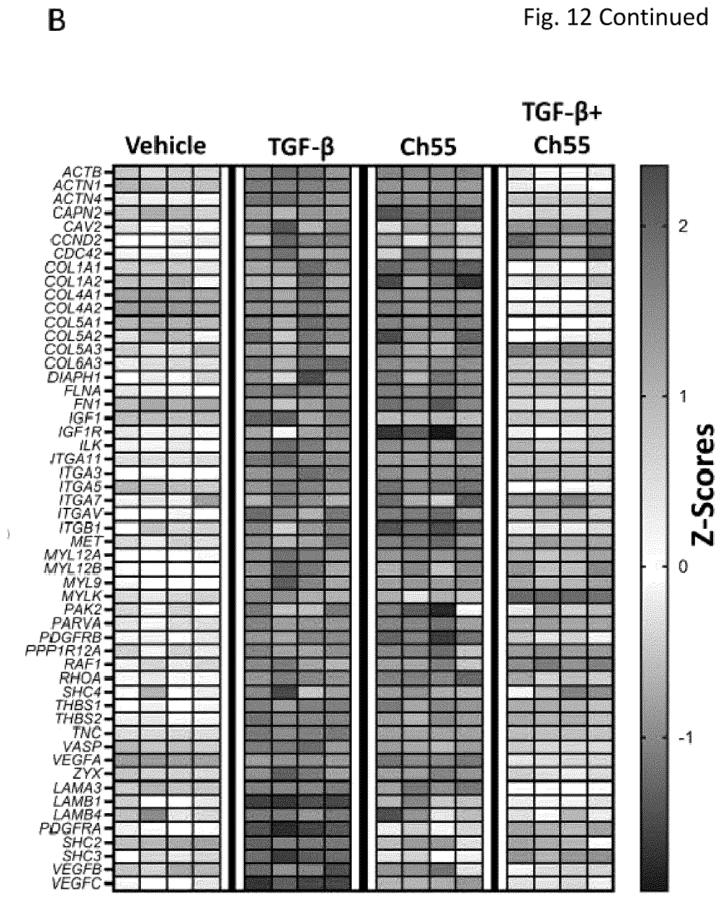

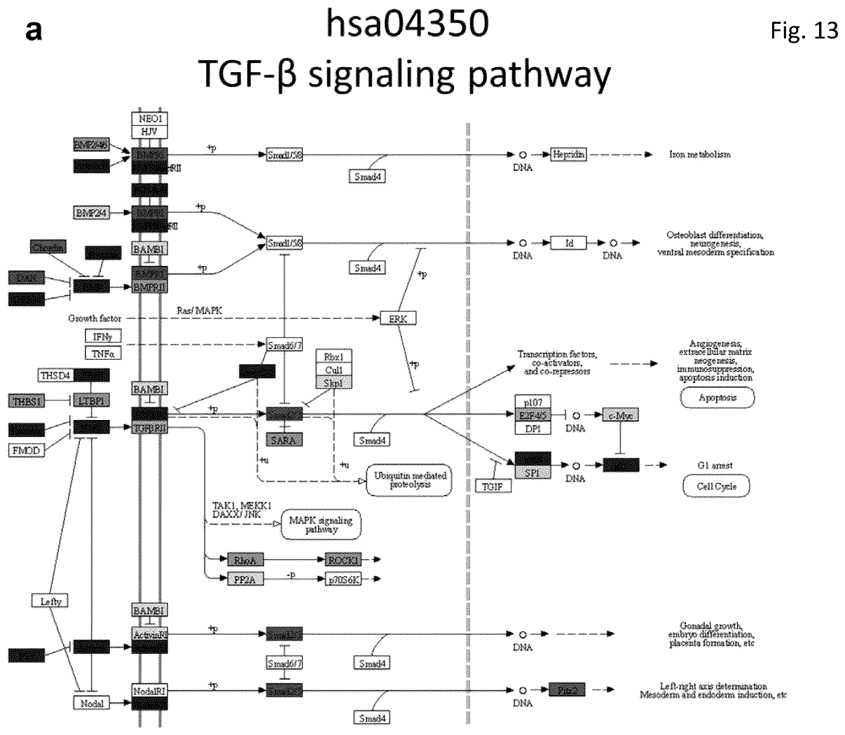

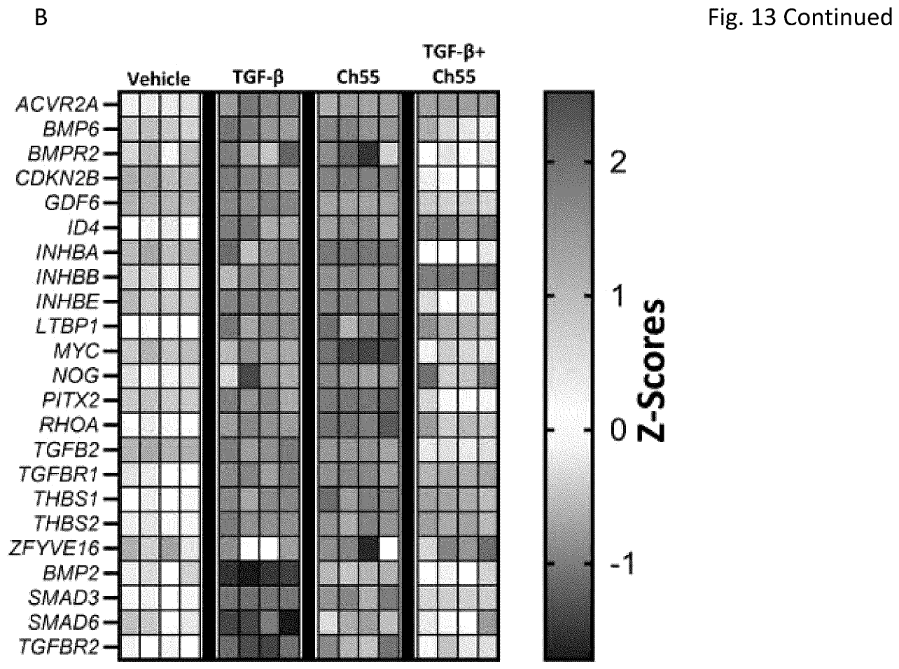

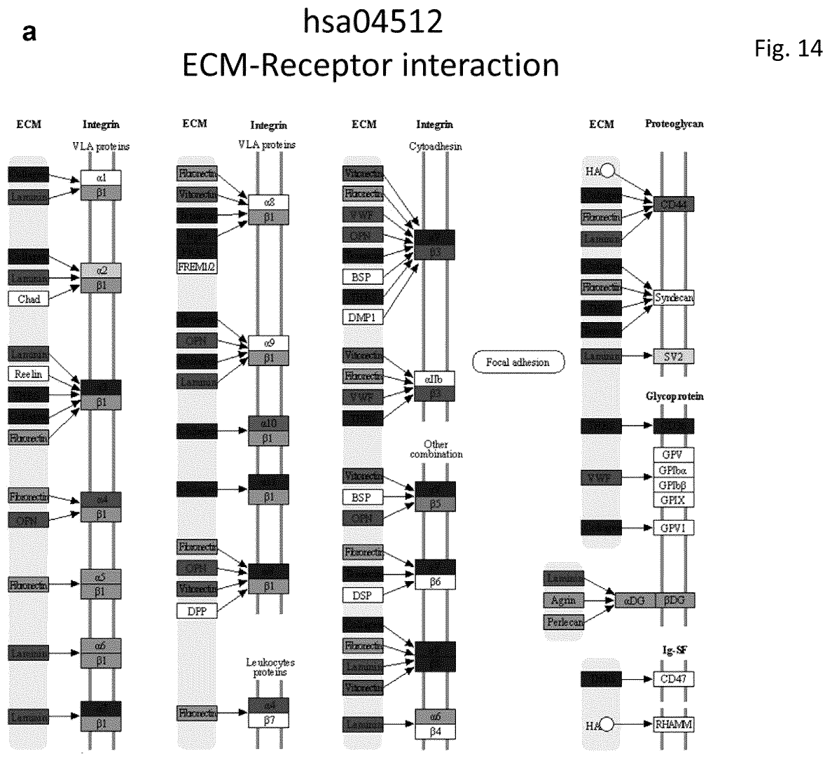

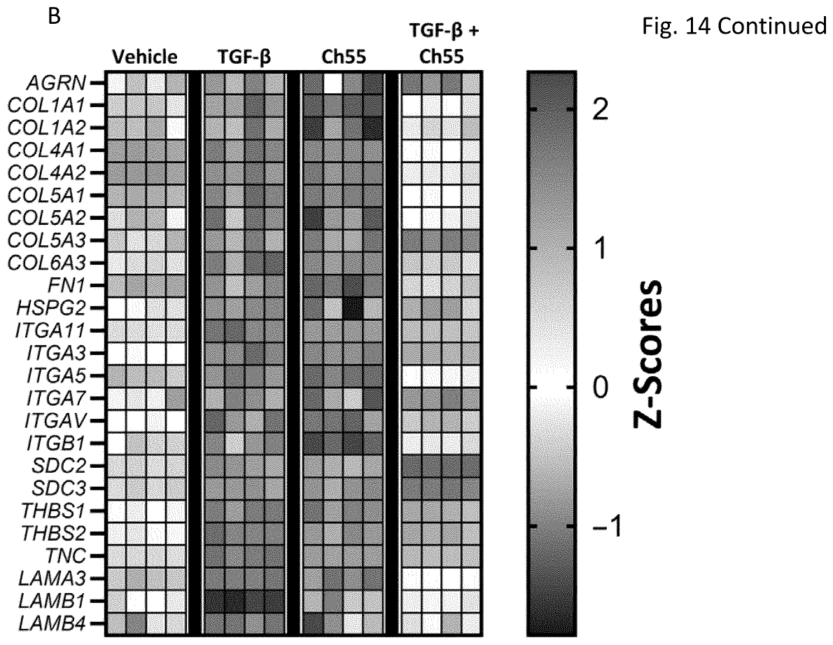

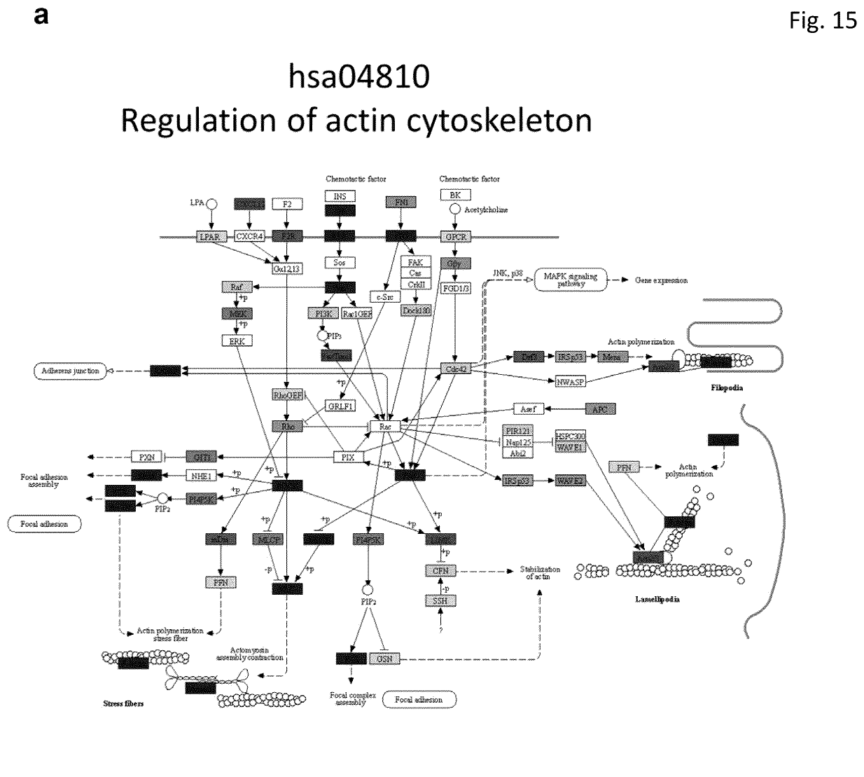

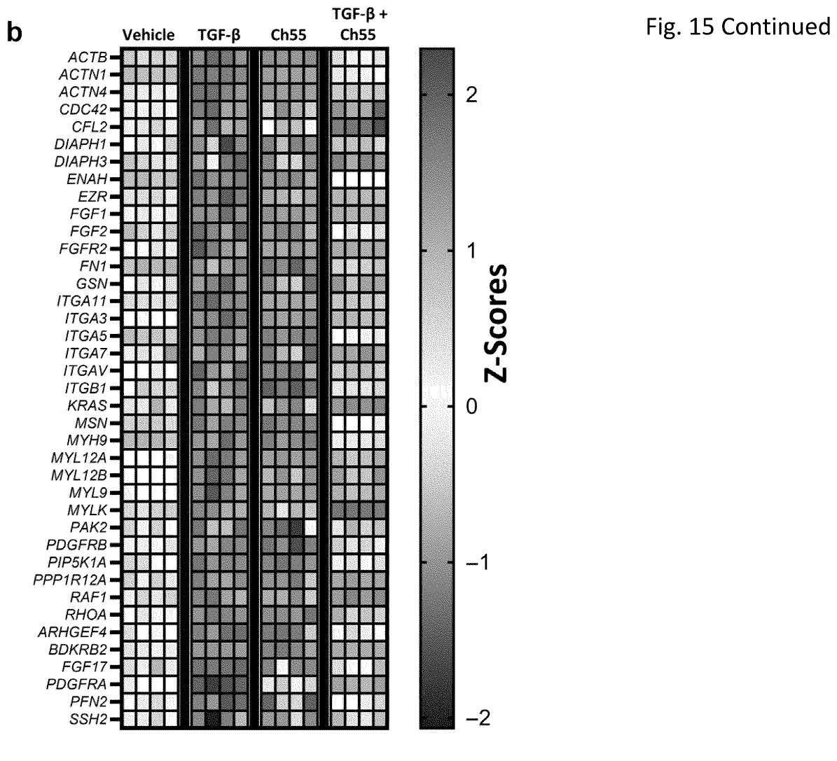

In order to more completely understand the effects of Ch55 stimulation, we performed bulk sequencing on RNA isolated from primary human foreskin fibroblasts treated for 24 hours with vehicle, 10 ng/mL TGF-01, 1,000 nM Ch55, or both 10 ng/mL TGF-β1 and 1,000 nM Ch55. Myofibroblast marker genes examined initially by qRT-PCR in FIG. 6 were confirmed to be significantly downregulated by Ch55 (FIG. 10), and many of the LRRC15⁺/Lrrc15⁺ signature-enriched genes (Table 1) initially used to query the CMap database were also found to be significantly downregulated by Ch55 (67/117, ˜57% of genes, Table 2). Additionally, many genes known to be targets or regulators of retinoic acid signaling exhibited broad upregulation by Ch55 (FIG. 11). Visualization of transcriptional profiles by two-dimensional principal component analysis (PCA, FIG. 3A) revealed clear separation among all treatment groups. Interestingly, relative placement of treatment groups on the PCA suggested that substantial groups of genes were differentially regulated both discordantly and concordantly by Ch55 and TGF-β1. We extracted the set of genes differentially expressed by Ch55 and regulated in the same direction by TGF-01 (TGF-β-mimicking signature, FIG. 3B). We also extracted the set of genes that was differentially expressed by Ch55, and the expression of which was regulated in the opposite direction by TGF-31 (TGF-θ-antagonizing signature, FIG. 3C). We then used goseq (Young et al., 2010) to query the KEGG database with the TGF-α-mimicking signature and the TGF-β-antagonizing signature. Extraction of KEGG annotations and associated pathways enriched in the TGF-α-mimicking signature (FIG. 3D) tended to refer to either general categories (e.g. “proteasome,” “cell cycle,” “phagosome”) or categories relevant to other specific cell types (e.g. “Epithelial cell signaling in H. pylori infection,” “Collecting duct acid secretion,” “Axon guidance”). In contrast, KEGG annotations for the TGF-β-antagonizing signature revealed several specific categories with clear relevance to fibroblast activation and fibrosis (FIG. 3E). We next computed normalized gene expression values for the DEGs extracted from the TGF-β-antagonizing signature and overlaid Ch55-induced relative expression effects on potentially fibrosis-relevant pathways of interest using Pathview Web (Luo et al., 2017). This analysis demonstrated broad reversal of TGF-β-mediated effects and pathway dysregulation for the KEGG terms “Focal adhesion” (FIG. 12), “TGF-θ signaling pathway” (FIG. 13), “ECM-Receptor interaction” (FIG. 14), and “Regulation of actin cytoskeleton” (FIG. 15). Taken together, these data suggest that Ch55 may be able to antagonize fibrosis by dysregulating cellular processes associated with fibroblast activation and cytoskeletal-ECM interactions.

TABLE 2
Directional effects of Ch55 treatment on the LRRC15⁺/Lrrc15⁺ gene
expression signature in primary human foreskin fibroblasts

| | Ch55 effect direction |
|---|---|
| | (From Ch55-Vehicle, |
| Gene Symbol | FDR P < 0.05) |
| ACTA2 | ↓ |
| ACTB | ↓ |
| ACTN1 | ↓ |
| ADAM12 | ↓ |
| AEBP1 | ↓ |
| AK1 | ←→ |
| ANGPTL2 | ↓ |
| ANTXR1 | ↓ |
| ARL4C | ↑ |
| ASPN | ↓ |
| BASP1 | ↑ |
| BGN | ↓ |
| BMP1 | ↓ |
| C1QTNF3 | ↓ |
| C1QTNF6 | ←→ |
| CALD1 | ↓ |
| CALU | ↓ |
| CKAP4 | ←→ |
| CNN2 | ↓ |
| CNN3 | ↑ |
| COL12A1 | ↓ |
| COL1A1 | ↓ |
| COL1A2 | ↓ |
| COL5A2 | ↓ |
| COL8A1 | ↓ |
| CREB3L1 | ↓ |
| CRTAP | ←→ |
| CSRP2 | ↓ |
| CTHRC1 | ↓ |
| CTNNB1 | ←→ |
| CTSK | ←→ |
| CXCL2 | ↑ |
| DAP | ←→ |
| EEF1G | ←→ |
| EFEMP2 | ←→ |
| FBLN2 | ↓ |
| FIBIN | ←→ |
| FKBP10 | ←→ |
| FKBP9 | ↓ |
| FN1 | ↓ |
| FNDC1 | ↓ |
| FOSB | ↓ |
| FOXP1 | ↓ |
| FSCN1 | ↓ |
| FZD1 | ←→ |
| GABARAP | ←→ |
| GAPDH | ↑ |
| GPC1 | ↑ |
| GPX7 | ↓ |
| HCFC1R1 | ↓ |
| INHBA | ↓ |
| ITGB5 | ↑ |
| ITGBL1 | ↓ |
| KDELR2 | ↓ |
| KDELR3 | ↓ |
| LOXL1 | ↓ |
| LOXL2 | ↑ |
| LRRC15 | ↑ |
| LUM | ↓ |
| MAGED1 | ↓ |
| MARCKSL1 | ↑ |
| MDK | ↑ |
| MFAP2 | ↓ |
| MIF | ←→ |
| MMP14 | ↑ |
| MMP19 | ↓ |
| MMP2 | ←→ |
| MORF4L2 | ↓ |
| MYH9 | ↓ |
| MYL9 | ↓ |
| NR4A2 | ←→ |
| NREP | ↓ |
| NXN | ↓ |
| OLFML2B | ←→ |
| OLFML3 | ↓ |
| P3H1 | ↓ |
| P3H4 | ↓ |
| P4HA3 | ↑ |
| P4HB | ←→ |
| PABPC1 | ↓ |
| PALLD | ↓ |
| PCSK5 | ←→ |
| PDGFRL | ↓ |
| PDLIM7 | ↓ |
| PMEPA1 | ↓ |
| POSTN | ↓ |
| PPIC | ←→ |
| PTK7 | ←→ |
| RAB31 | ←→ |
| RCN3 | ←→ |
| RGS3 | ←→ |
| SCARF2 | ↑ |
| SDC1 | ←→ |
| SFRP2 | ↓ |
| SKIL | ↓ |
| SLC39A14 | ←→ |
| SMCO4 | ←→ |
| SMIM3 | ↑ |
| SNHG18 | ↓ |
| SPARC | ↓ |
| SPATS2L | ↑ |
| SPON1 | ↑ |
| SRPX2 | ↑ |
| SSR3 | ←→ |
| SULF2 | ←→ |
| TAGLN | ↓ |
| THBS2 | ↓ |
| THY1 | ↓ |
| TMEM119 | ↑ |
| TMEM263 | ↓ |
| TNFAIP3 | ↓ |
| TPBG | ↑ |
| TPM1 | ↓ |
| TUBB2B | ↓ |
| VCAN | ↓ |
| WISP1 | ↑ |
| YWHAZ | ↓ |

### Ch55 Ameliorates Hypertrophic Scar Formation In Vivo

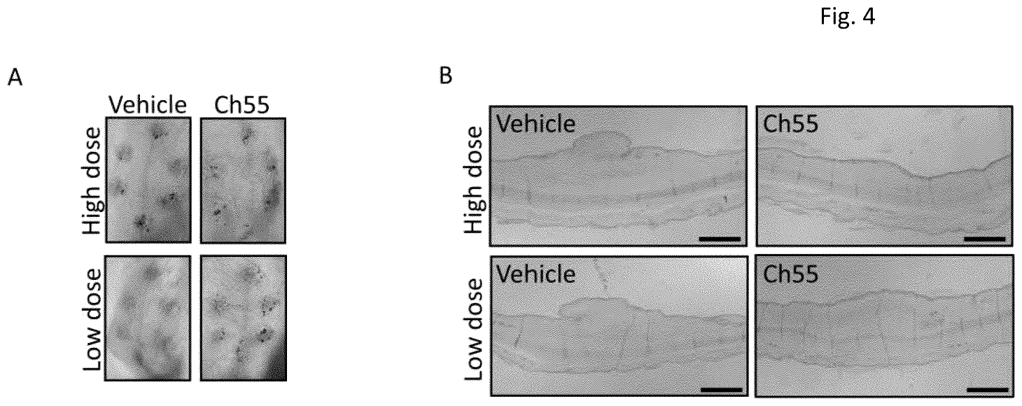

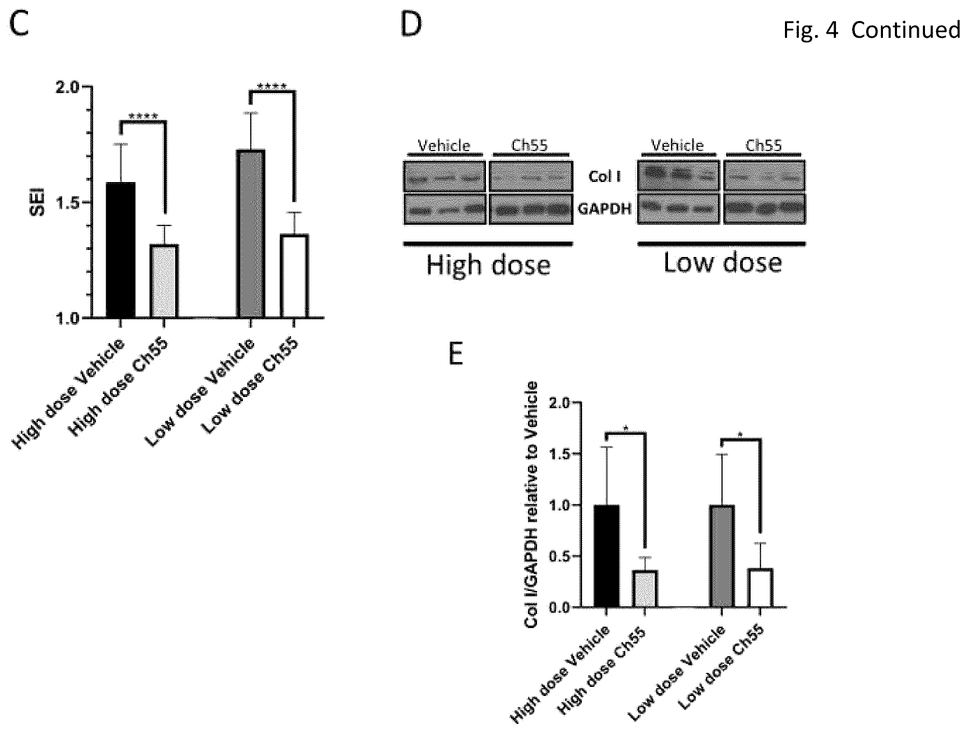

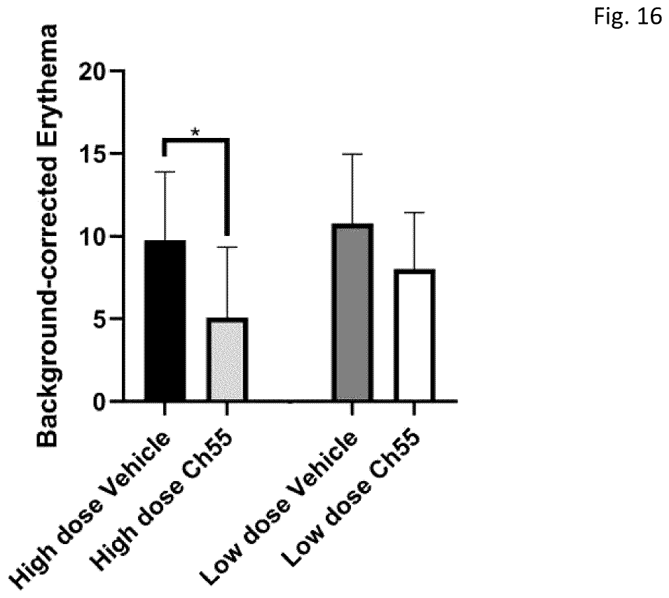

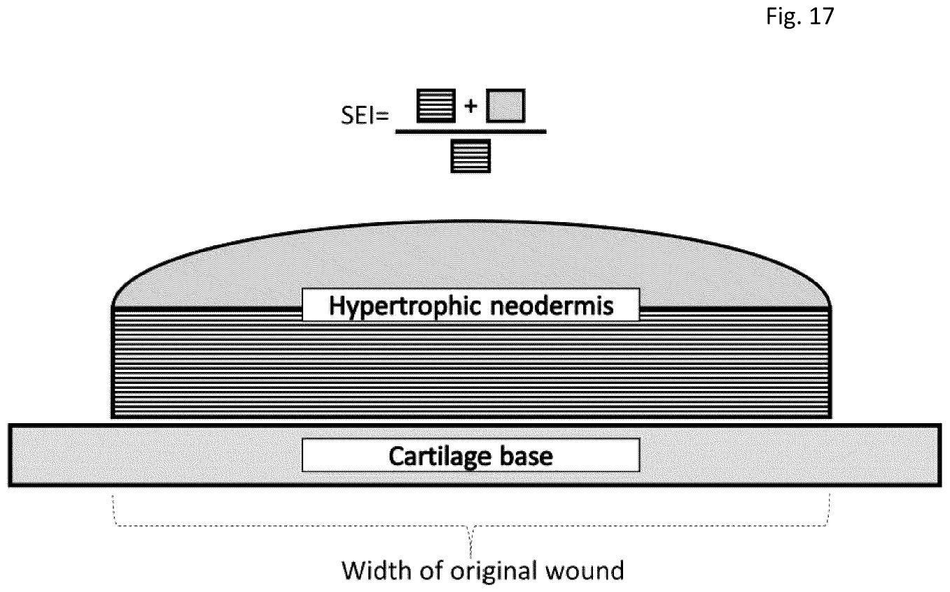

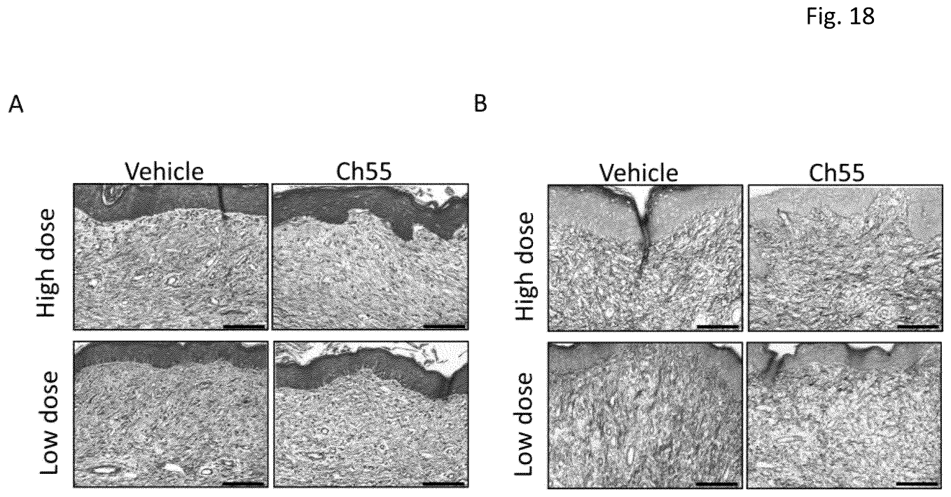

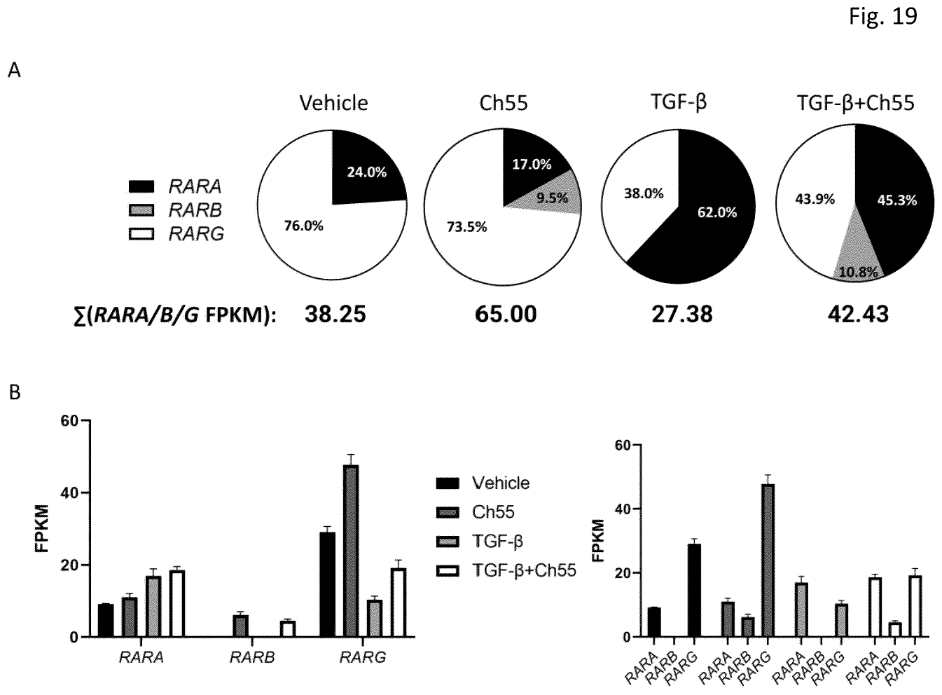

In order to determine whether the in vitro activity of Ch55 to antagonize fibroblast activation and fibrosis-associated pathways was accompanied by anti-fibrotic potential in vivo, we utilized a well-characterized model of excisional wound-induced hypertrophic scarring in the ears of New Zealand White rabbits. Once excisional wounds closed, we performed three sequential intradermal injections of either a high dose (10 μg) or low dose (2 μg) of Ch55, or their corresponding vehicles, into the developing scars and harvested scar tissues on post-operative day 28 (POD28, FIG. 4A). Spectroscopic measurements performed immediately prior to harvest and normalized to measurements of unwounded skin on the same ear revealed that administration of high dose Ch55 significantly decreased erythema in developed scars, while the decrease resulting from low dose Ch55 was not statistically significant (FIG. 16). Harvested scars were processed and analyzed to determine scar elevation index (SEI), a common quantitative histological measurement used to assess scar hypertrophy (FIG. 17). Administration of either high or low dose Ch55 was sufficient to significantly reduce resultant hypertrophy as assessed by SEI, relative to vehicle controls (FIG. 4B,C). Visualization of representative harvested tissues stained with modified Masson's Trichrome (FIG. 18A) and picrosirius red/fast green (FIG. 18B) suggested a possible decrease in collagen density of the scars of Ch55-treated wounds. Subsequently, detection by Western blot and quantification by densitometry confirmed that Ch55 administration at both high and low doses was sufficient to reduce the amount of type I collagen in the dermis at time of harvest (FIG. 4D,E). Taken together, these data demonstrated that dermal administration of Ch55 to developing scars was sufficient to limit hypertrophy and type I collagen deposition.

MATERIALS AND METHODS Materials Emetine dihydrochloride hydrate, homoharringtonine, Ch55 and panobinostat were acquired from Fisher Scientific (Waltham, MA). AZD-7762 was acquired from Selleck Chemicals (Houston, TX). For in vitro work, emetine dihydrochloride hydrate was dissolved in sterile H₂O at 50 mM. Homoharringtonine was dissolved in DMSO at 10 mg/mL. Panobinostat was dissolved in DMSO at 425 μM. Ch55 was dissolved in DMSO at 5 mM. AZD-7762 was dissolved in DMSO at 500 μM. Recombinant human TGF-β1 (Sigma-Aldrich) was dissolved in sterile H₂O at 50 g/mL. All-trans-retinoic acid (Thermo Scientific) was dissolved in DMSO at 10 mM. For in vivo work, Ch55 was dissolved at 1 mg/mL in 100% DMSO, and diluted 1:4 (v/v) into 1× sterile phosphate-buffered saline (PBS), for a final concentration of 200 μg/mL Ch55 (high dose). An aliquot of high dose Ch55 solution was then further diluted 1:4 in PBS, for a final concentration of 40 μg/mL Ch55 (low dose). Vehicle control for high dose Ch55 was prepared by diluting 100% DMSO 1:4 into PBS for a final formulation of 20% DMSO in 80% PBS. Vehicle control for low dose Ch55 was prepared by diluting 100% DMSO 1:24 into PBS for a final formulation of 4% DMSO in 96% PBS.

Cell culture Primary human neonatal foreskin fibroblasts were obtained from the Skin Biology and Diseases Resource-based Center at Northwestern University. Primary rabbit dermal fibroblasts were isolated from female New Zealand White rabbits via harvesting full-thickness skin biopsies from rabbit ears, digesting overnight in a solution of 5 mg/mL dispase, and subsequently mechanically separating dermis from epidermis. After discarding epidermal tissue, dermal tissue was minced with a razor blade, and fibroblasts were liberated through collagenase-mediated digestion for growth in culture. Human hypertrophic scar and keloid-derived tissues were collected from the discarded tissue of patients undergoing elective surgeries at Northwestern Memorial Hospital (Chicago, IL). Tissue collection was approved by the Institutional Review Board at Northwestern University. Fibroblasts from these tissues were isolated in the same manner as primary rabbit fibroblasts. Human fibroblasts and rabbit fibroblasts were cultured on tissue culture plastic (or, for immunofluorescence experiments, on glass coverslips) in DMEM+10% FBS and were serum starved in DMEM+0.1% FBS for 24 hours prior to drug treatments. When indicated, recombinant human TGF-β 1 was included in culture medium at a concentration of 10 ng/mL. Cultures were maintained in a humidified cell culture incubator at 37° C., 5% CO₂, and ambient O₂. For treatment with drugs and associated vehicle controls, the fraction of DMSO in the media was always maintained at <0.1% (v/v) in order to avoid confounding effects.

### Immunofluorescence

Cells cultured on glass coverslips were fixed in 4% paraformaldehyde for 30 minutes at room temperature. Cells were blocked in 10% normal goat serum and incubated overnight at 4° C. in solutions of the following primary antibodies: Rabbit monoclonal α-human α-SMA (1:500 dilution, catalog #192455, Cell Signaling Technology, Danvers M A) or mouse α-human COL1A1 (1:1000 dilution, catalog #M-38c, DSHB, Iowa City, IA). The following day, cells were incubated in 1:200 diluted solutions of goat-α-rabbit IgG or donkey-α-mouse IgG Alexafluor 488-conjugated secondary antibodies (A11034 or A21202, Invitrogen) containing Alexafluor 568-conjugated phalloidin (A12380, Invitrogen) for 2 hours in the dark at room temperature, before a 20 minute counterstain in 500 ng/mL DAPI. Coverslips were mounted onto slides in the dark using Fluoro Gel with DABCO (Electron Microscopy Sciences, Hatfield, PA) and imaged on an EVOS-FL imaging system (ThermoFisher).

RNA extraction and qRT-PCR

For RNA harvest, cultured cells were washed in cold phosphate-buffered saline (PBS), lysed in Tri reagent (Sigma-Aldrich, St. Louis, MO), and subjected to phenol/chloroform extraction and isopropanol precipitation, according to manufacturer's protocols. Remnant genomic DNA was digested and removed using the Turbo DNA-free kit (Ambion, Austin, TX). Total extracted RNA was reverse-transcribed into cDNA, according to manufacturer's instructions, using Superscript III reverse transcriptase (Invitrogen, Carlsbad, CA) and using random hexamers as primers. SYBR-based qRT-PCR was performed on a StepOnePlus Real Time PCR instrument (Applied Biosystems, Waltham, MA). GAPDH was used as an internal control in order to calculate ACtvalues, which were used for statistical comparisons. The AACₜ method was used to calculate relative fold changes, which were used to visualize expression differences among experimental groups. Primer sequences are listed in Table 3.

TABLE 3
Primer sequences for qRT-PCR

| Gene | Protein | Forward primer | Reverse primer |
|---|---|---|---|
| name | encoded | (5′-3′) | (5′-3′) |
| Human | Human | TGTTGCCATCAATG | CTCCACGACGTACT |
| GAPDH | GAPDH | ACCCCTT | CAGCG |
| | | SEQ ID NO: 1 | SEQ ID NO: 2 |
| Human | Human | CTATGAGGGCTATG | GCTCAGCAGTAGTA |
| ACTA2 | α-SMA | CCTTGCC | ACGAAGGA |
| | | SEQ ID NO: 3 | SEQ ID NO: 4 |
| Human | Human | ATCAACCGGAGGAA | CACCAGGACGACCA |
| COLIAI | collagen | TTTCCGT | GGTTTTC |
| | I α1 | SEQ ID NO: 5 | SEQ ID NO: 6 |
| Human | Human | AGTGACTGGGAAAC | GCTCTTGGCAAATC |
| MMP1 | MMP1 | CAGATGCTGA | TGGCCTGTAA |
| | | SEQ ID NO: 7 | SEQ ID NO: 8 |
| Human | Human | GCCCAGACCCAACT | TCTCCGTACATCTT |
| CCN2 | CTGF | ATGATTAG | CCTGTAGT |
| | | SEQ ID NO: 9 | SEQ ID NO: 10 |
| Human | Human | TGAAGACACACACA | AGTTCCAGGATGTC |
| SER- | PAI-1 | AAAGGT | GT AGT |
| PINE1 | | SEQ ID NO: 11 | SEQ ID NO: 12 |
| Human | Human | GTGGAAACCCACAA | ACAACTCCGGTGAC |
| TGFB1 | TGF-ß1 | CGAAATC | ATCAAA |
| | | SEQ ID NO: 13 | SEQ ID NO: 14 |
| Human | Human | AGGTTAAGAACAAG | GAGGCCGTCCATGA |
| CNN1 | calponin- | CTGGCCC | AGTTGT |
| | 1 | SEQ ID NO: 15 | SEQ ID NO: 16 |
| Human | Human | CACAAGGTGTGTGT | GGCTCATGCCATAG |
| TAGLN | SM22α | AAGGGTG | GAAGGAC |
| | | SEQ ID NO: 17 | SEQ ID NO: 18 |
| Human | Human | GCTCGTCCCTGATG | CGAAGGTCTGTCAC |
| EDN1 | endothe- | GATAAAG | CAATGT |
| | lin-1 | SEQ ID NO: 19 | SEQ ID NO: 20 |
| Human | Human | AAATTCGGTACATC | GGAAGGTTCAGGTT |
| IL6 | inter- | CTCGACGG | GTTTTCTGC |
| | leukin-6 | SEQ ID NO: 21 | SEQ ID NO: 22 |
| Rabbit | Rabbit | TGCTGTCCCTCTAT | GAAGGAATAGCCAC |
| ACTA2 | α-SMA | GCCTCT | GCTCAG |
| | | SEQ ID NO: 23 | SEQ ID NO: 24 |
| Rabbit | Rabbit | AGGTCATCCACGAC | GTGAGTTTCCCGTT |
| GAPDH | GAPDH | CACTTC | CAGCTC |
| | | SEQ ID NO: 25 | SEQ ID NO: 26 |

### RNA-Sequencing

Purified RNA harvested from primary cultured human foreskin fibroblasts pooled from three donors underwent TruSeq stranded mRNA-seq library preparation at the Northwestern University NUSeq core facility, followed by paired-end sequencing on an Illumina HiSeq 4000. Raw FASTQ files were imported into Galaxy (Jalili et al., 2020). Reads were trimmed using Trimmomatic (Bolger et al., 2014) utilizing default parameters, prior to aligning to the human genome (hg38 construction) using HISAT2 (Kim et al., 2019). Reads were assigned to genomic features using featureCounts (Liao et al., 2014), and differential expression among groups was determined, and principal component analysis constructed, using DESeq2 (Love et al., 2014) with multiple comparison adjustment performed by the Benjamini-Hochberg correction (Benjamini and Hochberg, 1995). Differentially-expressed genes were defined as false discovery rate-adjusted (FDR) P<0.05. Pathway analysis on KEGG databases (Kanehisa and Goto, 2000) were performed using goseq (Young et al., 2010) and Pathview Web (Luo et al., 2017). Z-scores were calculated from normalized count values and used to construct heatmaps. FPKM values were estimated using Stringtie (Pertea et al., 2015). Raw RNA-sequence data are available through the National Center for Biotechnology Information Sequence Read Archive accession number PRJNA921850, the contents of which is incorporated by reference in its entirety.

### Rabbit Ear Hypertrophic Scar Model

All animal experiments were approved prior to initiation by the Northwestern University Institutional Animal Care and Use Committee (IACUC). Rabbit experiments were performed in female New Zealand White rabbits (Envigo, Indianapolis, IN) of mass 2.5-3 kg. The hypertrophic scar model was performed as described in previous publications (Dolivo et al., Jia et al., 2017, Xie et al., 2020). Briefly, on each ear, six full-thickness circular excisional wounds of 7-mm diameter were created down to the perichondrium using biopsy punches, and excised tissue was removed gently using forceps. Wounds were covered with Tegaderm (3M Healthcare, St. Paul, MN) and wounds were allowed to re-epithelialize until post-operative day (POD) 12. Intradermal injections (-50 μL/injection using 30-gauge hollow needles) of Ch55 were performed to each wound on a randomized ear, and corresponding vehicle control injections were applied to each wound on the contralateral ear. Injections were performed on POD16, POD19, and POD22. On POD28, measurements of erythema were performed using a DermaLab Combo (CyberDerm, Broomall, PA) immediately prior to euthanasia and tissue harvest.

### Western Blot

Harvested scar tissues were incubated in 0.5M ammonium thiocyanate for 20 minutes, followed by mechanical separation of dermis and epidermis using a dissecting microscope. Isolated dermal tissues were minced with a razor blade, submerged in RIPA buffer, and homogenized in the presence of 2 mm zirconia beads using a MagNA Lyser (Roche, Basel, Switzerland). Alternatively, cell culture samples were detached from the culture surface by trypsinization, collected by centrifugation, and lysed in RIPA buffer. Protein concentrations of cell culture and tissue samples were determined using a DC protein assay (BioRad). Five micrograms of total protein were loaded onto polyacrylamide gels and subjected to SDS-PAGE before transfer to nitrocellulose membranes, blocking with 5% dry milk in TBS-T, and probing with target-specific primary antibodies overnight. Species-specific secondary antibody solutions were added to membranes, and chemiluminescent signal was developed with Amersham ECL Western blotting detection reagent using X-ray film.

Primary antibodies used were rabbit anti-α-SMA (1:1000, 19245S, Cell Signaling Technology), rabbit anti-collagen I (1:1000, ab34710, Abcam), mouse anti-collagen I (1:1000, C2456, Sigma-Aldrich), and mouse anti-GAPDH (1:5000, MA5-15738, Invitrogen). Secondary antibodies utilized were HRP goat anti-rabbit IgG(H+L) and horse anti-mouse IgG(H+L) (Vector Laboratories, Newark, CA), both used at 1:5000.

### Histology

Harvested tissues were fixed in 10% neutral-buffered formalin for 24 hours before serial dehydration and embedding in paraffin. Five-micron-thick sections were cut using a microtome and floated onto slides and dried overnight at 42° C. Slides were deparaffinized and rehydrated in xylene and serial ethanol. Tissue samples were stained with hematoxylin and eosin (H&E), Modified Masson's Trichrome stain (Scytek, Logan, UT), or picrosirius red/fast green according to standard protocols.

### Statistical Analysis

All quantitative visual figures were generated, and all statistical analyses performed, using Graphpad Prism 9 (Graphpad, San Diego, CA). Statistical comparisons between two groups were performed using two-tailed, unpaired Student's t-tests. For comparisons among more than two groups, one-way ANOVAs were performed with Dunnett's test for pairwise post-hoc comparisons to vehicle control, unless specified otherwise. All error bars represent population standard deviations. For all comparisons, *P<0.05, **P<0.01, ***P<0.001, ****P<0.0001.

### REFERENCES

ADDIN E N.REFLIST Abbasi S, Sinha S, Labit E, Rosin N L, Yoon G, Rahmani W, et al. Distinct regulatory programs control the latent regenerative potential of dermal fibroblasts during wound healing. Cell stem cell 2020; 27(3):396-412. e6.
Abergel R P, Meeker C A, Oikarinen H, Oikarinen A I, Uitto J. Retinoid modulation of connective tissue metabolism in keloid fibroblast cultures. Arch Dermatol 1985; 121(5):632-5.
Al Tanoury Z, Piskunov A, Andriamoratsiresy D, Gaouar S, Lutzing R, Ye T, et al. Genes involved in cell adhesion and signaling: a new repertoire of retinoic acid receptor target genes in mouse embryonic fibroblasts. Journal of Cell Science 2014; 127(3):521-33.
Ambinder A J, Norsworthy K, Hernandez D, Palau L, Paun B, Duffield A, et al. A Phase 1 Study of IRX195183, a RARa-Selective CYP26 Resistant Retinoid, in Patients With Relapsed or Refractory AML. Frontiers in Oncology 2020; 10: 587062.
Amiri N, Golin A P, Jalili R B, Ghahary A. Roles of cutaneous cell-cell communication in wound healing outcome: An emphasis on keratinocyte-fibroblast crosstalk. Experimental Dermatology 2022; 31(4):475-84.
Bahmer F A, Zaun H. Isotretinoin therapy for progressive systemic sclerosis. Archives of Dermatology 1985; 121(3):308-.
Beard R L, Duong T T, Teng M, Klein E S, Standevan A M, Chandraratna R A. Synthesis and biological activity of retinoic acid receptor-α specific amides. Bioorganic & medicinal chemistry letters 2002; 12(21):3145-8.
Benjamini Y, Hochberg Y. Controlling the false discovery rate: a practical and powerful approach to multiple testing. Journal of the Royal statistical society: series B (Methodological) 1995; 57(1):289-300.
Blume-Peytavi U, Fowler J, Kemeny L, Draelos Z, Cook-Bolden F, Dirschka T, et al. Long-term safety and efficacy of trifarotene 50 μg/g cream, a first-in-class RAR-γ selective topical retinoid, in patients with moderate facial and truncal acne. Journal of the European Academy of Dermatology and Venereology 2020; 34(1):166-73.
Bolger A M, Lohse M, Usadel B. Trimmomatic: a flexible trimmer for Illumina sequence data. Bioinformatics 2014; 30(15):2114-20.
Brown N, Cambruzzi J, Cox P J, Davies M, Dunbar J, Plumbley D, et al. Big data in drug discovery. Progress in medicinal chemistry 2018; 57:277-356.
Buechler M B, Pradhan R N, Krishnamurty A T, Cox C, Calviello A K, Wang A W, et al. Cross-tissue organization of the fibroblast lineage. Nature 2021; 593(7860):575-9.
Chen K, Henn D, Januszyk M, Barrera J A, Noishiki C, Bonham C A, et al. Disrupting mechanotransduction decreases fibrosis and contracture in split-thickness skin grafting. Science Translational Medicine 2022; 14 (645):eabj9152.
Cosio T, Di Prete M, Gaziano R, Lanna C, Orlandi A, Di Francesco P, et al. Trifarotene: a current review and perspectives in dermatology. Biomedicines 2021; 9(3):237.
Daly T J, Weston W L. Retinoid effects on fibroblast proliferation and collagen synthesis in vitro and on fibrotic disease in vivo. Journal of the American Academy of Dermatology 1986; 15(4):900-2.
Delany A M, Brinckerhoff C E. The synthetic retinoid (4-hydroxyphenyl) retinamide decreases collagen expression in vitro and in the tight-skin mouse. Arthritis and rheumatism 1993; 36(7):983-93.
Dolivo D, Rodrigues A, Sun L, Hou C, Li Y, Chung E, et al. Simvastatin cream alleviates dermal fibrosis in a rabbit ear hypertrophic scar model. Journal of Cosmetic Dermatology.
Dreno B, Kang S, Leyden J, York J. Update: Mechanisms of Topical Retinoids in Acne. Journal of drugs in dermatology: JDD 2022; 21(7):734-40.
Duscher D, Maan Z N, Wong V W, Rennert R C, Januszyk M, Rodrigues M, et al. Mechanotransduction and fibrosis. Journal of biomechanics 2014; 47(9):1997-2005.
Edward M, Gold J A, MacKIE R M. Modulation of melanoma cell adhesion to basement membrane components by retinoic acid. Journal of cell science 1989; 93(1):155-61.
Elder J T, Fisher G J, Zhang Q-Y, Eisen D, Krust A, Kastner P, et al. Retinoic acid receptor gene expression in human skin. Journal of investigative dermatology 1991; 96(4):425-33.
Fisher G J, Talwar H S, Xiao J-H, Datta S C, Reddy A P, Gaub M-P, et al. Immunological identification and functional quantitation of retinoic acid and retinoid X receptor proteins in human skin. Journal of Biological Chemistry 1994; 269(32):20629-35.
Hernandez D, Palau L, Norsworthy K, Anders N M, Alonso S, Su M, et al. Overcoming microenvironment-mediated protection from ATRA using CYP26-resistant retinoids. Leukemia 2020; 34(11):3077-81.
Hinz B, Phan S H, Thannickal V J, Galli A, Bochaton-Piallat M L, Gabbiani G. The myofibroblast: one function, multiple origins. Am J Pathol 2007; 170(6):1807-16.
Humphrey J D, Dufresne E R, Schwartz M A. Mechanotransduction and extracellular matrix homeostasis. Nature reviews Molecular cell biology 2014; 15(12):802-12.
Ikeda T, Ohtani T, Furukawa F. Vitamin A derivative etretinate improves bleomycin-induced scleroderma. Allergology International 2005; 54(3):419-25.
Ikeda T, Uede K, Hashizume H, Furukawa F. The vitamin A derivative etretinate improves skin sclerosis in patients with systemic sclerosis. Journal of dermatological science 2004; 34(1):62-6.
Jalili V, Afgan E, Gu Q, Clements D, Blankenberg D, Goecks J, et al. The Galaxy platform for accessible, reproducible and collaborative biomedical analyses: 2020 update. Nucleic acids research 2020; 48 (W1):W395-W402. Janssen de Limpens A. The local treatment of hypertrophic scars and keloids with topical retinoic acid. The British journal of dermatology 1980; 103(3):319-23.
Jetten A M, Anderson K, Deas M, Kagechika H, Lotan R, Rearick J, et al. New benzoic acid derivatives with retinoid activity: lack of direct correlation between biological activity and binding to cellular retinoic acid binding protein. Cancer research 1987; 47(13):3523-7.
Jia S, Xie P, Hong S J, Galiano R D, Mustoe T A. Local application of statins significantly reduced hypertrophic scarring in a rabbit ear model. Plastic and Reconstructive Surgery Global Open 2017; 5 (6).
Jumper N, Hodgkinson T, Arscott G, Har-Shai Y, Paus R, Bayat A. The aldo-keto reductase AKR1B10 is up-regulated in keloid epidermis, implicating retinoic acid pathway dysregulation in the pathogenesis of keloid disease. Journal of Investigative Dermatology 2016; 136(7):1500-12.
Kanehisa M, Goto S. KEGG: kyoto encyclopedia of genes and genomes. Nucleic acids research 2000; 28(1):27-30.
Kassir M, Karagaiah P, Sonthalia S, Katsambas A, Galadari H, Gupta M, et al. Selective RAR agonists for acne vulgaris: A narrative review. Journal of Cosmetic Dermatology 2020; 19(6):1278-83.
Kim D, Paggi J M, Park C, Bennett C, Salzberg S L. Graph-based genome alignment and genotyping with HISAT2 and HISAT-genotype. Nature biotechnology 2019; 37(8):907-15.
Kim R, Stern W. Retinoids and butyrate modulate fibroblast growth and contraction of collagen matrices. Investigative ophthalmology & visual science 1990; 31(6):1183-6.
Korfei M, Stelmaszek D, MacKenzie B, Skwarna S, Chillappagari S, Bach A C, et al. Comparison of the antifibrotic effects of the pan-histone deacetylase-inhibitor panobinostat versus the IPF-drug pirfenidone in fibroblasts from patients with idiopathic pulmonary fibrosis. PloS one 2018; 13 (11):e0207915.
Lee H J, Jang Y J. Recent understandings of biology, prophylaxis and treatment strategies for hypertrophic scars and keloids. Internationaljournal of molecular sciences 2018; 19(3):711.
Li X, Liu D, Ma Y, Du X, Jing J, Wang L, et al. Direct reprogramming of fibroblasts via a chemically induced XEN-like state. Cell stem cell 2017; 21(2):264-73. e7.
Li X, Wang S, Dai J, Yan L, Zhao S, Wang J, et al. Homoharringtonine prevents surgery-induced epidural fibrosis through endoplasmic reticulum stress signaling pathway. European Journal of Pharmacology 2017; 815:437-45.
Liao Y, Smyth G K, Shi W. featureCounts: an efficient general purpose program for assigning sequence reads to genomic features. Bioinformatics 2014; 30(7):923-30.
Love M I, Huber W, Anders S. Moderated estimation of fold change and dispersion for RNA-seq data with DESeq2. Genome biology 2014; 15(12):1-21.
Luo W, Pant G, Bhavnasi Y K, Blanchard Jr S G, Brouwer C. Pathview Web: user friendly pathway visualization and data integration. Nucleic acids research 2017; 45 (W1):W501-W8.
Mascharak S, Talbott H E, Januszyk M, Griffin M, Chen K, Davitt M F, et al. Multi-omic analysis reveals divergent molecular events in scarring and regenerative wound healing. Cell stem cell 2022; 29(2):315-27. e6.
Maurice P, Bunker C, Dowd P M. Isotretinoin in the treatment of systemic sclerosis. British Journal of Dermatology 1989; 121(3):367-74.
Mitchell J B, Choudhuri R, Fabre K, Sowers A L, Citrin D, Zabludoff S D, et al. In vitro and In vivo Radiation Sensitization of Human Tumor Cells by a Novel Checkpoint Kinase Inhibitor, AZD7762Radiosensitization by Chkl/2 Inhibition. Clinical cancer research 2010; 16(7):2076-84.
Mizutani H, Yoshida T, Nouchi N, Hamanaka H, Shimizu M. Topical tocoretinate improved hypertrophic scar, skin sclerosis in systemic sclerosis and morphea. The Journal of Dermatology 1999; 26(1):11-7.
Ohta A, Uitto J. Procollagen gene expression by scleroderma fibroblasts in culture. inhibition of collagen production and reduction of proa (i) and proal (III) collagen messenger RNA steady-state levels by retinoids. Arthritis & Rheumatism: Official Journal of the American College of Rheumatology 1987; 30(4):404-11.
Pakshir P, Noskovicova N, Lodyga M, Son D O, Schuster R, Goodwin A, et al. The myofibroblast at a glance. Journal of Cell Science 2020;133 (13):jcs227900.
Pertea M, Pertea G M, Antonescu C M, Chang T-C, Mendell J T, Salzberg S L. StringTie enables improved reconstruction of a transcriptome from RNA-seq reads. Nature biotechnology 2015; 33(3):290-5.
Redfern C, Todd C. Retinoic acid receptor expression in human skin keratinocytes and dermal fibroblasts in vitro. Journal of Cell Science 1992; 102(1):113-21.
Rees J L, Redfern C P. Expression of the a and p retinoic acid receptors in skin. Journal of investigative dermatology 1989; 93(6):818-20.
Rinkevich Y, Correa-Gallegos D, Ye H, Dasgupta B, Sardogan A, Ichijo R, et al. CD201⁺fascia progenitors choreograph injury repair. 2022.
Russo B, Brembilla N C, Chizzolini C. Interplay between keratinocytes and fibroblasts: a systematic review providing a new angle for understanding skin fibrotic disorders. Frontiers in immunology 2020; 11:648.
Russo B, Brembilla N C, Chizzolini C. Contribution of keratinocytes to dermal fibrosis. Current Opinion in Rheumatology 2022; 34(6):337-42.
Samarakoon R, Overstreet J M, Higgins P J. TGF-β signaling in tissue fibrosis: redox controls, target genes and therapeutic opportunities. Cellular signalling 2013; 25(1):264-8.

Sanchez AM, Shortrede JE, Vargas-Roig LM, Flamini MI. Retinoic acid induces nuclear FAK translocation and reduces breast cancer cell adhesion through Moesin, FAK, and Paxillin. Molecular and Cellular Endocrinology 2016; 430:1-11.
Scott LJ. Trifarotene: first approval. Drugs 2019; 79(17):1905-9.
Shima T, Yamamoto Y, Ikeda T, Furukawa F. A patient with localized scleroderma successfully treated with etretinate. Case Reports in Dermatology 2014; 6(3):200-6.
Subramanian A, Narayan R, Corsello S M, Peck D D, Natoli T E, Lu X, et al. A next generation connectivity map: L1000 platform and the first 1,000,000 profiles. Cell 2017;171(6):1437-52. e17.
Sun S-Y, Yue P, Dawson M I, Shroot B, Michel S, Lamph W W, et al. Differential effects of synthetic nuclear retinoid receptor-selective retinoids on the growth of human non-small cell lung carcinoma cells. Cancer Research 1997; 57(21):4931-9.
Sun Y, Dai J, Jiao R, Jiang Q, Wang J. Homoharringtonine inhibits fibroblasts proliferation, extracellular matrix production and reduces surgery-induced knee arthrofibrosis via PI3K/AKT/mTOR pathway-mediated apoptosis. Journal of Orthopaedic Surgery and Research 2021; 16(1):1-12.
Tan J, Thiboutot D, Popp G, Gooderham M, Lynde C, Del Rosso J, et al. Randomized phase 3 evaluation of trifarotene 50 μg/g cream treatment of moderate facial and truncal acne. Journal of the American Academy of Dermatology 2019; 80(6):1691-9.
Voigt A, Hartmann P, Zintl F. Differentiation, proliferation and adhesion of human neuroblastoma cells after treatment with retinoic acid. Cell Adhesion and Communication 2000; 7(5):423-40.
Walraven M, Hinz B. Therapeutic approaches to control tissue repair and fibrosis: Extracellular matrix as a game changer. Matrix Biol 2018;71-72:205-24.
Wynn TA. Cellular and molecular mechanisms of fibrosis. J Pathol 2008; 214(2):199-210.
Xiao R, Kanekura T, Yoshida N, Higashi Y, Yan KL, Fukushige T, et al. 9-Cis-retinoic acid exhibits antifibrotic activity via the induction of cyclooxygenase-2 expression and prostaglandin E2 production in scleroderma fibroblasts. Clinical and Experimental Dermatology: Experimental dermatology 2008; 33(4):484-90.
Xiao R, Yoshida N, Higashi Y, LU QJ, Fukushige T, Kanzaki T, et al. Retinoic acids exhibit anti-fibrotic activity through the inhibition of 5-lipoxygenase expression in scleroderma fibroblasts. The Journal of dermatology 2011; 38(4):345-53.
Xie P, Dolivo DM, Jia SX, Cheng XG, Salcido J, Galiano R D, et al. Liposome-encapsulated statins reduce hypertrophic scarring through topical application. Wound Repair and Regeneration 2020; 28(4):460-9.
Yang S, Xu M, Lee EM, Gorshkov K, Shiryaev SA, He S, et al. Emetine inhibits Zika and Ebola virus infections through two molecular mechanisms: inhibiting viral replication and decreasing viral entry. Cell discovery 2018; 4(1):1-14.
Ye J, Ge J, Zhang X, Cheng L, Zhang Z, He S, et al. Pluripotent stem cells induced from mouse neural stem cells and small intestinal epithelial cells by small molecule compounds. Cell research 2016; 26(1):34-45.
Young MD, Wakefield MJ, Smyth GK, Oshlack A. Gene ontology analysis for RNA-seq: accounting for selection bias. Genome biology 2010; 11(2):1-12.
Zhou T-B, Drummen GP, Qin Y-H. The controversial role of retinoic acid in fibrotic diseases: analysis of involved signaling pathways. International journal of molecular sciences 2013; 14(1):226-43.
Zouboulis CC. Retinoids-which dermatological indications will benefit in the near future? Skin Pharmacology and Physiology 2001; 14(5):303-15.

1. A method of antagonizing fibroblast activation in a subject in need thereof, the method comprising administering to the subject an effective amount of a retinoid.

2. The method of claim 1, wherein the fibroblast activation is in response to a wound or injury.

3. The method of claim 2, wherein the wound or injury results in the formation of scar tissue.

4. The method of claim 1, wherein the retinoid comprises a retinoic acid receptor agonist.

5. The method of claim 1, wherein the retinoid comprises CH 55.

6. The method of claim 1, wherein the retinoid comprises all-trans retinoic acid.

7. The method of claim 1, wherein the retinoid decreases expression of one or more of ACTA2, CNN1, CCN2, SERPINE1, TAGLN, EDN1, and IL6.

8. The method of claim 1, wherein the retinoid decreases expression of ACTA2, CNN1, CCN2, SERPINE1, TAGLN, EDN1, and IL6.

9. The method of claim 1, wherein the retinoid is administered dermally.

10. The method of claim 1, wherein the retinoid is administered by intradermal injection.

11. A method for treating scar formation, the method comprising administering an effective amount of a retinoid.

12. The method of claim 11, wherein the retinoid is administered dermally.

13. The method of claim 11, wherein the retinoid is administered by intradermal injection.

14. The method of claim 11, wherein the retinoid comprises a retinoic acid receptor agonist.

15. The method of claim 11, wherein the retinoid comprises CH 55.

16. The method of claim 11, wherein the retinoid comprises all-trans retinoic acid.

17. The method of claim 11, wherein the retinoid is administered following closure of a wound.

18. A method of antagonizing fibroblast activation, the method comprising administering an effective amount of a checkpoint kinase inhibitor.

19. The method of claim 18, wherein the checkpoint kinase inhibitor comprises AZD-7762.

20. The method of claim 18, wherein the checkpoint kinase inhibitor is administered dermally or by intradermal injection.


SEQUENCE LISTING (26 sequences)

SEQ ID NO: 1 - DNA, 21 bp, synthetic construct
  source 1..21: mol_type = other DNA
tgttgccatcaatgacccctt

SEQ ID NO: 2 - DNA, 19 bp, synthetic construct
  source 1..19: mol_type = other DNA
ctccacgacgtactcagcg

SEQ ID NO: 3 - DNA, 21 bp, synthetic construct
  source 1..21: mol_type = other DNA
ctatgagggctatgccttgcc

SEQ ID NO: 4 - DNA, 22 bp, synthetic construct
  source 1..22: mol_type = other DNA
gctcagcagtagtaacgaagga

SEQ ID NO: 5 - DNA, 21 bp, synthetic construct
  source 1..21: mol_type = other DNA
atcaaccggaggaatttccgt

SEQ ID NO: 6 - DNA, 21 bp, synthetic construct
  source 1..21: mol_type = other DNA
caccaggacgaccaggttttc

SEQ ID NO: 7 - DNA, 24 bp, synthetic construct
  source 1..24: mol_type = other DNA
agtgactgggaaaccagatgctga

SEQ ID NO: 8 - DNA, 24 bp, synthetic construct
  source 1..24: mol_type = other DNA
gctcttggcaaatctggcctgtaa

SEQ ID NO: 9 - DNA, 22 bp, synthetic construct
  source 1..22: mol_type = other DNA
gcccagacccaactatgattag

SEQ ID NO: 10 - DNA, 22 bp, synthetic construct
  source 1..22: mol_type = other DNA
tctccgtacatcttcctgtagt

SEQ ID NO: 11 - DNA, 20 bp, synthetic construct
  source 1..20: mol_type = other DNA
tgaagacacacacaaaaggt

SEQ ID NO: 12 - DNA, 19 bp, synthetic construct
  source 1..19: mol_type = other DNA
agttccaggatgtcgtagt

SEQ ID NO: 13 - DNA, 21 bp, synthetic construct
  source 1..21: mol_type = other DNA
gtggaaacccacaacgaaatc

SEQ ID NO: 14 - DNA, 20 bp, synthetic construct
  source 1..20: mol_type = other DNA
acaactccggtgacatcaaa

SEQ ID NO: 15 - DNA, 21 bp, synthetic construct
  source 1..21: mol_type = other DNA
aggttaagaacaagctggccc

SEQ ID NO: 16 - DNA, 20 bp, synthetic construct
  source 1..20: mol_type = other DNA
gaggccgtccatgaagttgt

SEQ ID NO: 17 - DNA, 21 bp, synthetic construct
  source 1..21: mol_type = other DNA
cacaaggtgtgtgtaagggtg

SEQ ID NO: 18 - DNA, 21 bp, synthetic construct
  source 1..21: mol_type = other DNA
ggctcatgccataggaaggac

SEQ ID NO: 19 - DNA, 21 bp, synthetic construct
  source 1..21: mol_type = other DNA
gctcgtccctgatggataaag

SEQ ID NO: 20 - DNA, 20 bp, synthetic construct
  source 1..20: mol_type = other DNA
cgaaggtctgtcaccaatgt

SEQ ID NO: 21 - DNA, 22 bp, synthetic construct
  source 1..22: mol_type = other DNA
aaattcggtacatcctcgacgg

SEQ ID NO: 22 - DNA, 23 bp, synthetic construct
  source 1..23: mol_type = other DNA
ggaaggttcaggttgttttctgc

SEQ ID NO: 23 - DNA, 20 bp, synthetic construct
  source 1..20: mol_type = other DNA
tgctgtccctctatgcctct

SEQ ID NO: 24 - DNA, 20 bp, synthetic construct
  source 1..20: mol_type = other DNA
gaaggaatagccacgctcag

SEQ ID NO: 25 - DNA, 20 bp, synthetic construct
  source 1..20: mol_type = other DNA
aggtcatccacgaccacttc

SEQ ID NO: 26 - DNA, 20 bp, synthetic construct
  source 1..20: mol_type = other DNA
gtgagtttcccgttcagctc